SEQUENCE LISTING (7 sequences)

SEQ ID NO: 1 - DNA, 2182 bp, Homo sapiens
  Bcl2-associated athanogene 4 (BAG4), Silencer of Death Domains (SODD)
aggtaagaggaaactccattggataaatggcgaggaaacgtatactccctcttaaggaacacggtgtcttccttcgtctccgggttcccgagaccccagagtcactgacctccgtccctcagctttcggggttcggcagcagaaggggcgggcccgggcctgggattggctggcgtcgtccgacccccttcgctgctctccattcgcaatcgcccgcgggcgcctgcgcgatgggtcggccgtggggagcggggcgggaagcgcttcagggcagcggatcccatgtcggccctgaggcgctcgggctacggccccagtgacggtccgtcctacggccgctactacgggcctgggggtggagatgtgccggtacacccacctccacccttatatcctcttcgccctgaacctccccagcctcccatttcctggcgggtgcgcgggggcggcccggcggagaccacctggctgggagaaggcggaggaggcgatggctactatccctcgggaggcgcctggccagagcctggtcgagccggaggaagccaccaggagcagccaccatatcctagctacaattctaactattggaattctactgcgagatctagggctccttacccaagtacatatcctgtaagaccagaattgcaaggccagagtttgaattcttatacaaatggagcgtatggtccaacataccccccaggccctggggcaaatactgcctcatactcaggggcttattatgcacctggttatactcagaccagttactccacagaagttccaagtacttaccgttcatctggcaacagcccaactccagtctctcgttggatctatccccagcaggactgtcagactgaagcaccccctcttagggggcaggttccaggatatccgccttcacagaaccctggaatgaccctgccccattatccttatggagatggtaatcgtagtgttccacaatcaggaccgactgtacgaccacaagaagatgcgtgggcttctcctggtgcttatggaatgggtggccgttatccctggccttcatcagcgccctcagcaccacccggcaatctctacatgactgaaagtacttcaccatggcctagcagtggctctccccagtcacccccttcacccccagtccagcagcccaaggattcttcatacccctatagccaatcagatcaaagcatgaaccggcacaactttccttgcagtgtccatcagtacgaatcctcggggacagtgaacaatgatgattcagatcttttggattcccaagtccagtatagtgctgagcctcagctgtatggtaatgccaccagtgaccatcccaacaatcaagatcaaagtagcagtcttcctgaagaatgtgtaccttcagatgaaagtactcctccgagtattaaaaaaatcatacatgtgctggagaaggtccagtatcttgaacaagaagtagaagaatttgtaggaaaaaagacagacaaagcatactggcttctggaagaaatgctaaccaaggaacttttggaactggattcagttgaaactgggggccaggactctgtacggcaggccagaaaagaggctgtttgtaagattcaggccatactggaaaaattagaaaaaaaaggattatgaaaggatttagaacaaagtggaagcctgttactaacttgaccaaagaacacttgatttggttaattaccctctttttgaaatgcctgttgatgacaagaagcaatacattccagctttcctttgattttatacttgaaaaactggcaaaggaatggaagaatattttagtcatgagttgttttcagttttcagacgaatgaatgtaataggaaactatggagttaccaatattgccaagtagactcactccttaaaaaatttatggatatctacaagctgcttcttaccagcaggagggaaacacacttcacacaacaggcttatcagaaacctaccagatgaaactggatataatctgagacaaacaggatgtgtttttttaaacatctggatatcttgtcacatttttgtacattgtgactgctttcaacatatacttcatgtgtaattatagcttagactttagccttcttggacttctgttttgttttgttatttgcagtttacaaatatagtattattctct

SEQ ID NO: 2 - protein, 457 aa, Homo sapiens
  Bcl2-associated athanogene 4 (BAG4), Silencer of Death Domains (SODD)
MSALRRSGYGPSDGPSYGRYYGPGGGDVPVHPPPPLYPLRPEPPQPPISWRVRGGGPAETTWLGEGGGGDGYYPSGGAWPEPGRAGGSHQEQPPYPSYNSNYWNSTARSRAPYPSTYPVRPELQGQSLNSYTNGAYGPTYPPGPGANTASYSGAYYAPGYTQTSYSTEVPSTYRSSGNSPTPVSRWIYPQQDCQTEAPPLRGQVPGYPPSQNPGMTLPHYPYGDGNRSVPQSGPTVRPQEDAWASPGAYGMGGRYPWPSSAPSAPPGNLYMTESTSPWPSSGSPQSPPSPPVQQPKDSSYPYSQSDQSMNRHNFPCSVHQYESSGTVNNDDSDLLDSQVQYSAEPQLYGNATSDHPNNQDQSSSLPEECVPSDESTPPSIKKIIHVLEKVQYLEQEVEEFVGKKTDKAYWLLEEMLTKELLELDSVETGGQDSVRQARKEAVCKIQAILEKLEKKGL

SEQ ID NO: 3 - DNA, 1814 bp, Homo sapiens
  ADP-ribosylation factor-1 (ARF1)
gcaaaaccaacgcctggctcggagcagcagcctctgaggtgtccctggccagtgtccttccacctgtccacaagcatggggaacatcttcgccaacctcttcaagggcctttttggcaaaaaagaaatgcgcatcctcatggtgggcctggatgctgcagggaagaccacgatcctctacaagcttaagctgggtgagatcgtgaccaccattcccaccataggcttcaacgtggaaaccgtggagtacaagaacatcagcttcactgtgtgggacgtgggtggccaggacaagatccggcccctgtggcgccactacttccagaacacacaaggcctgatcttcgtggtggacagcaatgacagagagcgtgtgaacgaggcccgtgaggagctcatgaggatgctggccgaggacgagctccgggatgctgtcctcctggtgttcgccaacaagcaggacctccccaacgccatgaatgcggccgagatcacagacaagctggggctgcactcactacgccacaggaactggtacattcaggccacctgcgccaccagcggcgacgggctctatgaaggactggactggctgtccaatcagctccggaaccagaagtgaacgcgacccccctccctctcactcctcttgccctctgctttactctcatgtggcaaacgtgcggctcgtggtgtgagtgccagaagctgcctccgtggtttggtcaccgtgtgcatcgcaccgtgctgtaaatgtggcagacgcagcctgcggccaggctttttatttaatgtaaatagtttttgtttccaatgaggcagtttctggtactcctatgcaatattactcagctttttttattgtaaaaagaaaaatcaactcactgttcagtgctgagaggggatgtaggcccatgggcacctggcctccaggagtcgctgtgttgggagagccggccacgcccttggcttagagctgtgttgaaatccattttggtggttggttttaacccaaactcagtgcattttttaaaatagttaagaatccaagtcgagaacacttgaacacacagaagggagaccccgcctagcatagatttgcagttacggcctggatgccagtcgccagcccagctgttcccctcgggaacatgaggtggtggtggcgcagcagactgcgatcaattctgcatggtcacagtagagatccccgcaactcgcttgtccttgggtcaccctgcattccatagccatgtgcttgtccctgtgctcccacggttcccaggggccaggctgggagcccacagccaccccactatgccgcaggccgccctacccaccttcaggcagcctatgggacgcaggccccatctgtccctcggtccgcgtgtggccagagtggtccgtcgtccccaacactcgtgctcgctcagacactttggcaggatgtctggggcctcaccagcaggagcgcgtgcaagccgggcaggcggtccacctagacccacagcccctcgggagcaccccacctctgtgtgtgatgtagctttctctccctcagcctgcaagggtccgatttgccatcgaaaaagacaacctctacttttttcttttgtattttgataaacactgaagctggagctgttaaatttatcttggggaaacctcagaactggtctatttggtgtcgtaggaacctcttactgctttcaatacacgattagtaatcaactgttttgtatacttgttttcagttttcatttcgacaaacaagcactgtaattatagctattagaataaaatctcttaactatt

SEQ ID NO: 4 - protein, 181 aa, Homo sapiens
  ADP-ribosylation factor-1 (ARF1)
MGNIFANLFKGLFGKKEMRILMVGLDAAGKTTILYKLKLGEIVTTIPTIGFNVETVEYKNISFTVWDVGGQDKIRPLWRHYFQNTQGLIFVVDSNDRERVNEAREELMRMLAEDELRDAVLLVFANKQDLPNAMNAAEITDKLGLHSLRHRNWYIQATCATSGDGLYEGLDWLSNQLRNQK

SEQ ID NO: 5 - DNA, 3921 bp, Homo sapiens
  Ephrin Receptor A2 (EPHA2), Epithelial Cell Receptor Protein-Tyrosine Kinase (ECK)
cggaagttgcgcgcaggccggcgggcgggagcggacaccgaggccggcgtgcaggcgtgcgggtgtgcgggagccgggctcggggggatcggaccgagagcgagaagcgcggcatggagctccaggcagcccgcgcctgcttcgccctgctgtggggctgtgcgctggccgcggccgcggcggcgcagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtaacaactttgagctcaactttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccttccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcaaggtgcacgaattccagacgctgtccccggagggatctggcaacttggcggtgattggcggcgtggctgtcggtgtggtcctgcttctggtgctggcaggagttggcttctttatccaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatctgagcctcgacagggcctggagccccatcggccaagaatacttgaagaaacagagtggcctccctgctgtgccatgctgggccactggggactttatttatttctagttctttcctccccctgcaacttccgctgaggggtctcggatgacaccctggcctgaactgaggagatgaccagggatgctgggctgggccctctttccctgcgagacgcacacagctgagcacttagcaggcaccgccacgtcccagcatccctggagcaggagccccgccacagccttcggacagacatataggatattcccaagccgaccttccctccgccttctcccacatgaggccatctcaggagatggagggcttggcccagcgccaagtaaacagggtacctcaagccccatttcctcacactaagagggcagactgtgaacttgactgggtgagacccaaagcggtccctgtccctctagtgccttctttagaccctcgggccccatcctcatccctgactggccaaacccttgctttcctgggcctttgcaagatgcttggttgtgttgaggtttttaaatatatattttgtactttgtggagagaatgtgtgtgtgtggcagggggccccgccagggctggggacagagggtgtcaaacattcgtgagctggggactcagggaccggtgctgcaggagtgtcctgcccatgccccagtcggccccatctctcatccttttggataagtttctattctgtcagtgttaaagattttgttttgttggacatttttttcgaatcttaatttattattttttttatatttattgttagaaaatgacttatttctgctctggaataaagttgcagatgattcaaaccg

SEQ ID NO: 6 - protein, 976 aa, Homo sapiens
  Ephrin Receptor A2 (EPHA2), Epithelial Cell Receptor Protein-Tyrosine Kinase (ECK)
MELQAARACFALLWGCALAAAAAAQGKEVVLLDFAAAGGELGWLTHPYGKGWDLMQNIMNDMPIYMYSVCNVMSGDQDNWLRTNWVYRGEAERNNFELNFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 7 - protein, 200 aa, Artificial Sequence
  Description of Artificial Sequencepoly-Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

(19) United States
(12) Patent Application Publication
(10) Pub. No.: US 2005/0009033 A1
(43) Pub. Date: Jan. 13, 2005

(54) Breast cancer genes

(75) Inventors: Joe W. Gray, San Francisco, CA (US); Richard M. Neve, San Francisco, CA (US); Frank McCormick, San Francisco, CA (US); Jennifer Yeh, San Francisco, CA (US); Koei Chin, Foster City, CA (US); Madhu Macrae, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/616,403

(22) Filed: Jul. 8, 2003

(51) Int. Cl. C12Q 1/68; G01N 33/574

(52) U.S. Cl. 435/6; 435/7.23

(57) ABSTRACT

This invention is based upon the discovery that EPHA2, BAG4, and ARF1 are amplified and overexpressed in cancer. The present invention therefore provides methods, reagents, and kits for diagnosing and treating breast cancer.

### BACKGROUND OF THE INVENTION

Curative treatment of individual metastatic breast cancers is likely to require an battery of therapeutic agents targeted against the diversity of deregulated molecular pathways that contribute to the cancer phenotype. Although agents that successfully target genes involved in such pathways have been developed, e.g., herceptin, these agents are not effective against all breast cancers. Accordingly, there is a need to develop agents that target other genes. This invention addresses that need.

### BRIEF SUMMARY OF THE INVENTION

The current invention is based on the discovery of EPHA2, BAG4, or ARF1 nucleic acid and protein sequences are amplified and over-expressed in breast cancer. Accordingly, the invention provides methods to detect breast cancer or a propensity to develop cancer, to monitor the efficacy of a breast cancer treatment, and/or of using the sequence for prognostic applications. The invention also provides methods of identifying inhibitors of EPHA2, BAG4, or ARF1 as well as methods of treating breast cancer, e.g., by inhibiting the expression and/or activity of EPHA2, BAG4, or ARF1.

In one aspect, the invention provides a method of detecting breast cancer cells in a biological sample, e.g., breast tissue, from a patient, typically a human. The method comprising detecting overexpression of EPHA2, BAG4, or ARF1 in the biological sample, thereby detecting tumor tissue in the biological sample.

In one embodiment, overexpression of EPHA2, BAG4, or ARF1 is detected using an antibody that selectively binds to EPHA2, BAG4, or ARF1. Often, the amount of EPHA2, BAG4, or ARF1 polypeptide is quantified by immunoassay. In another embodiment, detecting overexpression of EPHA2, BAG4, or ARF1 comprises detecting the activity of EPHA2, BAG4, or ARF1.

In an alternative embodiment, detecting overexpression of EPHA2, BAG4, or ARF1 comprises detecting an mRNA that encodes EPHA2, BAG4, or ARF1. Often, the mRNA is detected using an amplification reaction.

In one embodiment, the patient is undergoing a therapeutic regimen to treat breast cancer. In another embodiment, the patient is suspected of having metastatic breast cancer.

In another aspect, the present invention provides a method of detecting the presence of a breast cancer cell in a biological sample, e.g., breast tissue, from a patient, typically a human. The method comprises providing the biological sample and detecting an increase in copy number of EPHA2, BAG4, or ARF1 relative to a normal control, thereby detecting the presence of breast cancer. In one embodiment, the detecting step comprises contacting a sample comprising a EPHA2, BAG4, or ARF1 gene with a probe that selectively hybridizes to the gene under conditions in which a stable hybridization complex is formed and detecting the hybridization complex. Often, the contacting step includes a step of amplifying the gene in an amplification reaction. In one embodiment, the amplification reaction is a polymerase chain reaction.

In one embodiment, the patient is undergoing a therapeutic regimen to treat breast cancer. In another embodiment, the patient is suspected of having metastatic breast cancer.

In another aspect, the invention provides a method of identifying a compound that inhibits EPHA2, BAG4, or ARF1 activity, the method comprising contacting the compound with a EPHA2, BAG4, or ARF1 polypeptide and detecting a decrease in the activity of the EPHA2, BAG4, or ARF1 polypeptide. In one embodiment, the polypeptide is linked to a solid phase. In another embodiment, the EPHA2, BAG4, or ARF1 polypeptide is expressed in a cell. Additionally, the EPHA2, BAG4, or ARF1 gene may be amplified in the cell compared to normal.

In another aspect, the invention provides a method of inhibiting proliferation of a breast cancer cell in which EPHA2, BAG4, or ARF1 is amplified and overexpressed, the method comprising the step of contacting the breast cancer cell with a therapeutically effective amount of an inhibitor of EPHA2, BAG4, or ARF1. Typically, the inhibitor is identified as described herein.

In one embodiment, the inhibitor is an antibody. In another embodiment, the inhibitor is a small molecule.

In another aspect, the present invention provides a method of identifying an inhibitor of EPHA2, BAG4, or ARF1 comprising the steps of: (i) administering a test compound to a mammal having breast cancer or to a cell sample isolated from the mammal (ii) comparing the level of an EPHA2, BAG4, or ARF1 polynucleotide or polypeptide sequence in the cell or mammal to the level of gene expression of the sequence in a control cell sample or mammal; and (iii) selecting a test compound that decreases the level of the EPHA2, BAG4, or ARF1 polynucleotide or polypeptide relative to the control.

In one embodiment, EPHA2, BAG4, or ARF1 is amplified and overexpressed in breast cancer cells from the mammal.

In another embodiment, the control sample is a normal cell from the mammal with breast cancer or from a normal mammal.

In another aspect, the present invention provides a method for treating a mammal, typically a human, having breast cancer comprising administering a compound identified using a method described herein.

In another aspect, the present invention provides a pharmaceutical composition for treating a mammal having breast cancer, the composition comprising a compound identified using a method described herein and a physiologically acceptable excipient.

### BRIEF DESCRIPTION OF THE DRAWINGS

FIG. 1 depicts frequencies of copy number gains (positive values) and losses (negative values) in 152 human breast tumors (upper panel) and 66 breast cancer cell lines (lower panel). Frequency is displayed according to genomic location with chromosome 1pter to the left and chromosome 22qter and X to the right. Vertical lines indicate chromosome boundaries.

FIG. 2 is a graphical representation of gene copy number plotted against gene expression.

FIG. 3 show the results of a western analysis of whole-cell lysates from human breast cancer cell lines. Levels of EPHA2 and ERBB3 were determined.

### DETAILED DESCRIPTION OF THE INVENTION

Introduction

The present invention provides methods, reagents, and kits for diagnosing breast cancer, for prognostic uses, and for treating cancer. The invention is based upon the discovery that EPHA2, BAG4, or ARF1 polynucleotide and polypeptides are overexpressed in breast cancer cells.

EPHA2

Ephrin Receptor A2 (EPHA2), also called Epithelial Cell Receptor Protein-Tyrosine Kinase (ECK), is a member of the EPH and EPH-related receptor subfamily of receptor protein-tyrosine kinases. It has been shown to be overexpressed in breast cancer (Zelinski et al., Cancer Res. 61:2301-2306, 2001). In some embodiments of the current invention, detection of overexpression of EPHA2 nucleic acid and/or polypeptide sequences can be used as an indicator of the prognosis for breast cancer patients. EPHA2 polynucleotide and polypeptides sequences are known. Exemplary human EPHA2 nucleic acid sequences are available under the reference sequence NM_{—}004431 and the GenBank accession numbers M59371 and BC037166. An exemplary polypeptide sequence is available under the accession number NP_{—}004422.

BAG4

Bcl2-associated athanogene 4 (BAG4), which is also known as Silencer of Death Domains (SODD) is involved in apoptosis. Tumor Necrosis Factor Receptor-1 (TNFR1) and several other members of the TNF receptor superfamily, such as DR3, contain intracellular death domains and are capable of triggering apoptosis when activated by their respective ligands. However, TNFR1 self-associates and signals independently of ligand when overexpressed. Jiang, et al., (Science 283: 543-546, 1999) suggested the existence of a cellular mechanism to protect against ligand-independent signaling by TNFR1 and other death domain receptors. Using a yeast 2-hybrid assay with DR3 as bait, these authors identified a cDNA encoding a protein that they designated ‘silencer of death domains’ (SODD). The predicted 457-amino acid SODD protein migrates as a doublet of 60 kD on Western blots of mammalian cell extracts. Co-immunoprecipitation studies revealed that SODD is associated with TNFR1 in vivo. TNF treatment of cells released SODD from TNFR1, permitting the recruitment of proteins such as TRADD and TRAF2 to the active TNFR1 signaling complex.

BAG1 binds the ATPase domains of Hsp70 and Hsc70, modulating their chaperone activity. Takayama, et al., (J. Biol. Chem. 274: 781-786, 1999) identified cDNAs corresponding to BAG4 and three other BAG1-like proteins. These authors suggested that interactions with various BAG family proteins allow opportunities for specification and diversification of Hsp70/Hsc70 chaperone functions.

It has been shown that pancreatic cancer cells are resistant to TNFα-mediated apoptosis and that SODD is overexpressed in pancreatic cancer relative to normal (Ozawa, et al, Biochem. Biophys. Res. Commun. 271: 409-413, 2000). Other gastrointestinal cancers (e.g., liver, esophagus, stomach, and colon) showed no increased SODD expression.

BAG4 sequences are known. Exemplary human nucleic acid sequences are available, e.g., under the reference sequence NM_{—}004874 and Genbank accession numbers AF111116 and AF095194. Exemplary human polypeptide sequences are available under the accession numbers AAD05226, AAD16123, NP_{—}004865; and 095429.

ARF1

ADP-ribosylation factor-1 (ARF1) is a small guanine nucleotide-binding protin that is a member of the RAS superfamily. ARF1 is involved in vesicular transport and activates phospholipase D. These functions are tied to its ability to reversibly associate with membranes, interact with phospholipids, and the hydrolysis of GTP. ARF1 sequences are known. Bobak et al. (Proc. Nat. Acad. Sci. 86:6101-6105, 1989) cloned two ARF cDNAs, ARF1 and ARF3, from a human cerebellum library. Based on deduced amino acid sequences and patterns of hybridization of cDNA and oligonucleotide probes with mammalian brain poly(A)+ RNA, human ARF1 is the homolog of bovine ARF1. Lee et al. (J. Biol. Chem. 267: 9028-9034, 1992) found that human ARF1 is identical to its bovine counterpart, has a distinctive pattern of tissue and developmental expression, and is encoded by an mRNA of approximately 1.9 kb.

Exemplary human nucleic acid sequences are available, e.g., under the reference sequence NM_{—}001658 and Genbank accession numbers M84326, M36340, AF055002, and AF052179. Exemplary human polypeptide sequences are available under the accession numbers AAA35511, AAA35512, AAA35552, P32889, AAC09356, AAC28623, NP_{—}001649, AAH09247, and AAH10429.

The ability to detect breast cancer cells by virtue of detecting an increased level of a EPHA2, BAG4, or ARF1 nucleic acid or polypeptide sequence is useful for any of a large number of applications. For example, an increased level of EPHA2, BAG4, or ARF1 in cells of patient can be used, alone or in combination with other diagnostic methods, to diagnose breast cancer in the patient or to determine the propensity of a patient to develop breast cancer. The detection of EPHA2, BAG4, or ARF1 sequences can also be used to monitor the efficacy of a cancer treatment. For example, the level of a EPHA2, BAG4, or ARF1 polypeptide or polynucleotide after an anti-cancer treatment is compared to the level before the treatment. A decrease in the level of the EPHA2, BAG4, or ARF1 polypeptide or polynucleotide after the treatment indicates efficacious treatment.

An increased level or diagnostic presence of EPHA2, BAG4, or ARF1 can also be used to influence the choice of anti-cancer treatment, where, for example, the increased level of EPHA2, BAG4, or ARF1 directly correlates with the aggressiveness of the cancer and accordingly, the selection of anti-cancer therapy.

In addition, the ability to detect breast cancer cells can be useful to monitor the number or location of cancer cells in a patient, in vivo or in vitro, for example, to monitor the progression of the cancer over time. In addition, the level of EPHA2, BAG4, or ARF1 can be statistically correlated with the efficacy of particular anti-cancer therapies or with observed prognostic outcomes, thereby allowing the development of databases based on which a statistically-based prognosis, or a selection of the most efficacious treatment, can be made in view of a particular level or diagnostic presence of EPHA2, BAG4, or ARF1.

The present invention also provides methods of identifying inhibitors of EPHA2, BAG4, or ARF1 and methods for treating cancer. In certain embodiments, the proliferation is inhibited in a breast cancer cell that has an increase in copy number of EPHA2, BAG4, or ARF1 and overexpresses the sequence. The proliferation is decreased by, for example, contacting the cell with an inhibitor of EPHA2, BAG4, or ARF1 transcription or translation, or an inhibitor of the activity of EPHA2, BAG4, or ARF1. Such inhibitors include, but are not limited to, antibodies, small molecule inhibitors, antisense polynucleotides, ribozymes, and dominant negative EPHA2, BAG4, or ARF1 polynucleotides or polypeptides.

Definitions

The term “EPHA2”, “BAG4”, or “ARF1” refers to nucleic acid and polypeptide polymorphic variants, alleles, mutants, and interspecies homologues that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 20, 50, 100, 200, 500, 1000, or more amino acids, to a EPHA2, BAG4, or ARF1 sequence of SEQ ID NO:2; 4, or 6; (2) bind to antibodies, e.g., polyclonal antibodies, raised against an immunogen comprising an amino acid sequence of SEQ ID NO:2,4, or 6, or 8, or conservatively modified variants thereof; (3) specifically hybridize under stringent hybridization conditions to a EPHA2, BAG4, or ARF1 nucleic acid sequence of SEQ ID NO:1, 3, or 5, or conservatively modified variants thereof; or (4) or have a nucleic acid sequence that has greater than about 90%, preferably greater than about 96%, 97%, 98%, 99%, or higher nucleotide sequence identity, preferably over a region of over a region of at least about 30, 50, 100, 200, 500, 1000, or more nucleotides, to SEQ ID NO:1, 3, or 5; or (5) have at least 25, often 50, 75, 100, 150, 200, 250, 300, 350, 400 or more contiguous amino acid of SEQ ID NO:2, 4, or 6; or at least 25, often 50, 75, 100, 150, 200, 250, 300, 350, 400, 500, or more contiguous nucleotides of SEQ ID NO:1, 3, or 5. A EPHA2, BAG4, or ARF1 polynucleotide or polypeptide sequence is typically from a human, but may be from other mammals, but not limited to, a non-human primate, a rodent, e.g., a rat, mouse, or hamster; a cow, a pig, a horse, a sheep, or other mammal. A “EPHA2”, “BAG4”, or “ARF1” polypeptide and a “EPHA2”, “BAG4”, or “ARF1” polynucleotide include both naturally occurring or recombinant forms.

A “full length” EPHA2, BAG4, or ARF protein or nucleic acid refers to a EPHA2, BAG4, or ARF polypeptide or polynucleotide sequence, or a variant thereof, that contains all of the elements normally contained in one or more naturally occurring, wild type EPHA2, BAG4, or ARF polynucleotide or polypeptide sequences. The “full length” may be prior to, or after, various stages of post-translation processing or splicing, including alternative splicing.

“Biological sample” as used herein is a sample of biological tissue or fluid that contains nucleic acids or polypeptides, e.g., of a breast cancer protein, polynucleotide or transcript. Such samples are typically from humans, but include tissues isolated from non-human primates, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues.

“Providing a biological sample” means to obtain a biological sample for use in methods described in this invention. Most often, this will be done by removing a sample of cells from a patient, but can also be accomplished by using previously isolated cells (e.g., isolated by another person, at another time, and/or for another purpose), or by performing the methods of the invention in vivo. Archival tissues, having treatment or outcome history, will be particularly useful.

The “level of EPHA2, BAG4, or ARF1 mRNA” in a biological sample refers to the amount of mRNA transcribed from an EPHA2, BAG4, or ARF1 gene that is present in a cell or a biological sample. The mRNA generally encodes a functional EPHA2, BAG4, or ARF1 protein, although mutations may be present that alter or eliminate the function of the encoded protein. A “level of EPHA2, BAG4, or ARF1 mRNA” need not be quantified, but can simply be detected, e.g., a subjective, visual detection by a human, with or without comparison to a level from a control sample or a level expected of a control sample.

The “level of EPHA2, BAG4, or ARF1 protein or polypeptide” in a biological sample refers to the amount of polypeptide translated from EPHA2, BAG4, or ARF1 mRNA that is present in a cell or biological sample. The polypeptide may or may not have EPHA2, BAG4, or ARF1 protein activity. A “level of EPHA2, BAG4, or ARF1 protein” need not be quantified, but can simply be detected, e.g., a subjective, visual detection by a human, with or without comparison to a level from a control sample or a level expected of a control sample.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor & Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A variety of references disclose such nucleic acid analogs, including, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

Other analogs include peptide nucleic acids (PNA) which are peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into the breast cancer nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence. Diagnosis or prognosis may be based at the genomic level, or at the level of RNA or protein expression.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec −2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

The phrase “functional effects” in the context of assays for testing compounds that modulate activity of a breast cancer protein includes the determination of a parameter that is indirectly or directly under the influence of the breast cancer protein or nucleic acid, e.g., a functional, physical, or chemical effect, such as the ability to decrease breast cancer. It includes ligand binding activity; cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of breast cancer cells. “Functional effects” include in vitro, in vivo, and ex vivo activities.

By “determining the functional effect” is meant assaying for a compound that increases or decreases a parameter that is indirectly or directly under the influence of a breast cancer protein sequence, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the breast cancer protein; measuring binding activity or binding assays, e.g. binding to antibodies or other ligands, and measuring cellular proliferation. Determination of the functional effect of a compound on breast cancer can also be performed using breast cancer assays known to those of skill in the art such as an in vitro assays, e.g., cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of breast cancer cells. The functional effects can be evaluated by many means known to those skilled in the art, e.g., microscopy for quantitative or qualitative measures of alterations in morphological features, measurement of changes in RNA or protein levels for breast cancer-associated sequences, measurement of RNA stability, identification of downstream or reporter gene expression (CAT, luciferase, β-gal, GFP and the like), e.g., via chemiluminescence, fluorescence, colorimetric reactions, antibody binding, inducible markers, and ligand binding assays.

“Inhibitors” or “modulators” of EPHA2, BAG4, or ARF polynucleotide and polypeptide sequences are used to refer to inhibitory molecules or compounds identified using in vitro and in vivo assays of EPHA2, BAG4, or ARF polynucleotide and polypeptide sequences. Inhibitors are compounds that, e.g., bind to, partially or totally block activity, decrease, prevent, delay activation, inactivate, desensitize, or down regulate the activity or expression of EPHA2, BAG4, or ARF proteins, e.g., antagonists. Inhibitors include antisense or siRNA, genetically modified versions of breast cancer proteins, e.g., versions with altered activity, as well as naturally occurring and synthetic ligands, antagonists, agonists, antibodies, small chemical molecules and the like. Such assays for inhibitors and activators include, e.g., expressing the breast cancer protein in vitro, in cells, or cell membranes, applying putative modulator compounds, and then determining the functional effects on activity, as described above.

Samples or assays comprising EPHA2, BAG4, or ARF proteins that are treated with a potential inhibitor are compared to control samples without the inhibitor, to examine the extent of inhibition. Control samples (untreated with inhibitors) are assigned a relative protein activity value of 100%. Inhibition of a EPHA2, BAG4, or ARF polypeptide is achieved when the activity value relative to the control is about 80%, preferably 50%, more preferably 25-0%.

The phrase “changes in cell growth” refers to any change in cell growth and proliferation characteristics in vitro or in vivo, such as formation of foci, anchorage independence, semi-solid or soft agar growth, changes in contact inhibition and density limitation of growth, loss of growth factor or serum requirements, changes in cell morphology, gaining or losing immortalization, gaining or losing tumor specific markers, ability to form or suppress tumors when injected into suitable animal hosts, and/or immortalization of the cell. See, e.g., Freshney, Culture of Animal Cells a Manual of Basic Technique pp. 231-241 (3^{rd} ed. 1994).

“Tumor cell” refers to precancerous, cancerous, and normal cells in a tumor.

“Cancer cells,” “transformed” cells or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, nonmorphological changes, and/or malignancy (see, Freshney, Culture of Animal Cells a Manual of Basic Technique (3^{rd} ed. 1994)).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

Identification of Breast Cancer-Associated Sequences in a Sample from a Patient

In one aspect of the invention, the expression levels of EPHA2, BAG4 or ARF1 are determined in different patient samples for which diagnostic or prognostic information is desired. That is, normal tissue (e.g., normal breast or other tissue) may be distinguished from cancerous or metastatic cancerous tissue of the breast; or breast cancer tissue or metastatic breast cancerous tissue can be compared with tissue samples of breast and other tissues from other patients, e.g., surviving cancer patients.

General Recombinant DNA Methods

This invention relies on routine techniques in the field of recombinant genetics. Basic texts disclosing the general methods of use in this invention include Sambrook & Russell, Molecular Cloning, A Laboratory Manual (3rd Ed, 2001); Kriegler, Gene Transfer and Expression: A Laboratory Manual (1990); and Current Protocols in Molecular Biology (Ausubel et al., eds., 1994-1999). Methods that are used to produce EPHA2, BAG4 or ARF1 for use in the invention may also be employed to produce protein ligands or polypeptides that modulate ligand binding to the receptor, for use in the invention.

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

Cloning Methods for the Isolation of Nucleotide Sequences

In general, the nucleic acid sequences encoding EPHA2, BAG4, or ARF1 and related nucleic acid sequence homologs are cloned from cDNA and genomic DNA libraries by hybridization with a probe, or isolated using amplification techniques with oligonucleotide primers. For example, sequences are typically isolated from mammalian nucleic acid (genomic or cDNA) libraries by hybridizing with a nucleic acid probe, the sequence of which can be derived from SEQ ID NOS:1, 3, or 5.

Amplification techniques using primers can also be used to amplify and isolate nucleic acids from DNA or RNA (see, e.g., section “detection of polynucleotides”, below). Suitable primers for amplification of specific sequences can be designed using principles well known in the art (see, e.g., Dieffenfach & Dveksler, PCR Primer: A Laboratory Manual (1995)). These primers can be used, e.g., to amplify either the full length sequence or a probe, typically varying in size from ten to several hundred nucleotides, which is then used to identify EPHA2, BAG4, or ARF1 polynucleotides.

Nucleic acids encoding EPHA2, BAG4, or ARF1 can also be isolated from expression libraries using antibodies as probes. Such polyclonal or monoclonal antibodies can be raised using the sequence of SEQ ID NOs:2, 4, or 6.

Synthetic oligonucleotides can also be used to construct EPHA2, BAG4, or ARF1 genes for use as probes or for expression of protein. This method is performed using a series of overlapping oligonucleotides usually 40-120 bp in length, representing both the sense and nonsense strands of the gene. These DNA fragments are then annealed, ligated and cloned. Alternatively, amplification techniques can be used with precise primers to amplify a specific subsequence of the nucleic acid. The specific subsequence is then ligated into an expression vector.

The nucleic acid encoding EPHA2, BAG4, or ARF1 is typically cloned into intermediate vectors before transformation into prokaryotic or eukaryotic cells for replication and/or expression. These intermediate vectors are typically prokaryote vectors, e.g., plasmids, or shuttle vectors.

Optionally, nucleic acids encoding chimeric proteins comprising EPHA2, BAG4, or ARF1 or domains thereof can be made according to standard techniques. For example, a domain such as ligand binding domain can be covalently linked to a heterologous protein., e.g., green fluorescent protein, luciferase, or β-gal.

Detection of Polynucleotides

Typically, the level of a EPHA2, BAG4, or ARF1 polynucleotide or polypeptide will be detected in a biological sample. A “biological sample” refers to a cell or population of cells or a quantity of tissue or fluid from an animal. Most often, the sample has been removed from an animal, but the term “biological sample” can also refer to cells or tissue analyzed in vivo, i.e., without removal from the animal. Typically, a “biological sample” will contain cells from the animal, but the term can also refer to noncellular biological material, such as noncellular fractions of blood, saliva, or urine, that can be used to measure the cancer-associated polynucleotide or polypeptide levels. Numerous types of biological samples can be used in the present invention, including, but not limited to, a tissue biopsy, a blood sample, a buccal scrape, a saliva sample, or a nipple discharge.

As used herein, a “tissue biopsy” refers to an amount of tissue removed from an animal for diagnostic analysis. In a patient with cancer, tissue may be removed from a tumor, allowing the analysis of cells within the tumor. “Tissue biopsy” can refer to any type of biopsy, such as needle biopsy, fine needle biopsy, surgical biopsy, etc.

Detection of Copy Number

In one embodiment, the presence of cancer is evaluated by determining the copy number of cancer-associated genes, i.e., the number of DNA sequences in a cell encoding EPHA2, BAG4, or ARF1. Methods of evaluating the copy number of a particular gene are well known to those of skill in the art, and include, inter alia, hybridization and amplification based assays.

Hybridization-Based Assays

Any of a number of hybridization based assays can be used to detect the copy number of EPHA2, BAG4, or ARF1 in the cells of a biological sample. One such method is by Southern blot. In a Southern blot, genomic DNA is typically fragmented, separated electrophoretically, transferred to a membrane, and subsequently hybridized to a cancer-associated polynucleotide-specific probe. Comparison of the intensity of the hybridization signal from the probe for the target region with a signal from a control probe for a region of normal genomic DNA (e.g., a nonamplified portion of the same or related cell, tissue, organ, etc.) provides an estimate of the relative copy number of the cancer-associated gene. Southern blot methodology is well known in the art and is described, e.g., in Ausubel et al., or Sambrook et al., supra.

An alternative means for determining the copy number of EPHA2, BAG4, or ARF1 in a sample is by in situ hybridization, e.g., fluorescence in situ hybridization, or FISH. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments.

The probes used in such applications are typically labeled, e.g., with radioisotopes or fluorescent reporters. Preferred probes are sufficiently long, e.g., from about 50, 100, or 200 nucleotides to about 1000 or more nucleotides, so as to specifically hybridize with the target nucleic acid(s) under stringent conditions.

In numerous embodiments, “comparative probe” methods, such as comparative genomic hybridization (CGH), are used to detect EPHA2, BAG4, or ARF1 gene amplification. In comparative genomic hybridization methods, a “test” collection of nucleic acids is labeled with a first label, while a second collection (e.g., from a healthy cell or tissue) is labeled with a second label. The ratio of hybridization of the nucleic acids is determined by the ratio of the first and second labels binding to each fiber in an array. Differences in the ratio of the signals from the two labels, e.g., due to gene amplification in the test collection, is detected and the ratio provides a measure of the EPHA2, BAG4, or ARF1 gene copy number.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc.

Amplification-Based Assays

In another embodiment, amplification-based assays are used to measure the copy number of EPHA2, BAG4, or ARF1. In such an assay, the EPHA2, BAG4, or ARF1 nucleic acid sequences act as a template in an amplification reaction (e.g., Polymerase Chain Reaction, or PCR). In a quantitative amplification, the amount of amplification product will be proportional to the amount of template in the original sample. Comparison to appropriate controls provides a measure of the copy number of the cancer-associated gene. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.). The known nucleic acid sequences for EPHA2, BAG4, or ARF1 (see, e.g., SEQ ID NO:1, 3, or 7) is sufficient to enable one of skill to routinely select primers to amplify any portion of the gene.

In preferred embodiments, a TaqMan based assay is used to quantify the cancer-associated polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, literature provided by Perkin-Elmer, e.g., www2.perkin-elmer.com).

Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Detection of mRNA Expression

Direct Hybridization-Based Assays

Methods of detecting and/or quantifying the level of EPHA2, BAG4, or ARF1 gene transcripts (mRNA or cDNA made therefrom) using nucleic acid hybridization techniques are known to those of skill in the art. For example, one method for evaluating the presence, absence, or quantity of EPHA2, BAG4, or ARF1 polynucleotides involves a Northern blot: mRNA is isolated from a given biological sample, electrophoresed and transferred from the gel to a nitrocellulose membrane. Labeled EPHA2, BAG4, or ARF1 probes are then hybridized to the membrane to identify and/or quantify the mRNA.

Amplification-Based Assays

In another embodiment, a EPHA2, BAG4, or ARF1 transcript is detected using amplification-based methods (e.g., RT-PCR). RT-PCR methods are well known to those of skill (see, e.g., Ausubel et al., supra). Preferably, quantitative RT-PCR, e.g., a Taqman assay, is used, thereby allowing the comparison of the level of mRNA in a sample with a control sample or value.

Gene expression levels of EPHA2, BAG4, or ARF1 can also be analyzed by techniques known in the art, e.g., dot blotting, in situ hybridization, RNase protection, probing DNA microchip arrays, and the like. In one embodiment, high density oligonucleotide analysis technology (e.g., GeneChip™) is used to identify EPHA2, BAG4, or ARF1 sequences.

Expression in Prokaryotes and Eukaryotes

To obtain high level expression of a cloned gene or nucleic acid, such as cDNAs encoding EPHA2, BAG4, or ARF1, one typically subclones a EPHA2, BAG4, or ARF1 nucleic acid into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook & Russell, supra, Ausubel et al, supra. Bacterial expression systems for expressing the EPHA2, BAG4, or ARF1 protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the EPHA2, BAG4, or ARF1-encoding nucleic acid in host cells. A typical expression cassette thus contains a promoter operably linked to the nucleic acid sequence encoding a EPHA2, BAG4, or ARF1 and signals required for efficient polyadenylation of the transcript, ribosome binding sites, and translation termination. The nucleic acid sequence encoding a EPHA2, BAG4, or ARF1 may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a EPHA2, BAG4, or ARF1-encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of EPHA2, BAG4, or ARF1 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook and Russell., supra). It is only necessary that the particular genetic engineering procedure used be capable of successfully introducing at least one gene into the host cell capable of expressing a EPHA2, BAG4, or ARF1.

After the expression vector is introduced into the cells, the transfected cells are cultured under conditions favoring expression of EPHA2, BAG4, or ARF1, which is recovered from the culture using standard techniques (see, e.g., Scopes, Protein Purification: Principles and Practice (1982); U.S. Pat. No. 4,673,641; Ausubel et al., supra; and Sambrook et al., supra).

Production of Antibodies and Immunological Detection EPHA2, BAG4, or ARF1

Antibodies can also be used to detect EPHA2, BAG4, or ARF1 or can be assessed in the methods of the invention for the ability to inhibit EPHA2, BAG4, or ARF1. A general overview of the applicable technology can be found in Harlow & Lane, Antibodies: A Laboratory Manual (1988) and Harlow & Lane, Using Antibodies (1999). Methods of producing polyclonal and monoclonal antibodies that react specifically with EPHA2, BAG4, or ARF1 are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Such antibodies can be used for therapeutic and diagnostic or prognostic applications, e.g., in the treatment and/or detection of breast cancer.

In one embodiment, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for EPHA2, BAG4, or ARF1, or a fragment thereof, the other one is for any other antigen, and preferably for a cell-surface protein or receptor or receptor subunit, preferably one that is tumor specific. Alternatively, tetramer-type technology may create multivalent reagents.

In one embodiment, the antibodies to the EPHA2, BAG4, or ARF1 protein are capable of reducing or eliminating a biological function of EPHA2, BAG4, or ARF1, as is described below. That is, the addition of anti-EPHA2, BAG4, or ARF1 antibodies (either polyclonal or preferably monoclonal) to breast cancer tissue (or cells containing breast cancer) may reduce or eliminate the breast cancer. Generally, at least a 25% decrease in activity, growth, size or the like is preferred, with at least about 50% being particularly preferred and about a 95-100% decrease being especially preferred.

Often, the antibodies to the EPHA2, BAG4, or ARF1 proteins are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

Human antibodies can also be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

By immunotherapy is meant treatment of breast cancer with an antibody raised against EPHA2, BAG4, or ARF1 proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In another embodiment, the anti-EPHA2, BAG4, or ARF1 antibody is conjugated to an effector moiety. The effector moiety can be any number of molecules, including labelling moieties such as radioactive labels or fluorescent labels, or can be a therapeutic moiety. In one aspect the therapeutic moiety is a small molecule that modulates the activity of the breast cancer protein. In another aspect the therapeutic moiety modulates the activity of molecules associated with or in close proximity to the breast cancer protein. The therapeutic moiety may inhibit enzymatic activity such as kinase activity associated with breast cancer.

In a preferred embodiment, the therapeutic moiety can also be a cytotoxic agent. In this method, targeting the cytotoxic agent to breast cancer tissue or cells, results in a reduction in the number of afflicted cells, thereby reducing symptoms associated with breast cancer. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against breast cancer proteins, or binding of a radionuclide to a chelating agent that has been covalently attached to the antibody. Targeting the therapeutic moiety to transmembrane breast cancer proteins not only serves to increase the local concentration of therapeutic moiety in the breast cancer afflicted area, but also serves to reduce deleterious side effects that may be associated with the therapeutic moiety.

In another embodiment, the protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell.

EPHA2, BAG4, or ARF1 or a fragment thereof may be used to produce antibodies specifically reactive with EPHA2, BAG4, or ARF1. For example, a recombinant EPHA2, BAG4, or ARF1 or an antigenic fragment thereof, is isolated as described herein. Recombinant protein is the preferred immunogen for the production of monoclonal or polyclonal antibodies. Alternatively, a synthetic peptide derived from the sequences disclosed herein and conjugated to a carrier protein can be used as an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Typically, polyclonal antisera with a titer of 10⁴ or greater are selected and tested for their cross reactivity against non-EPHA2, BAG4, or ARF1 proteins or even other related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

Once EPHA2, BAG4, or ARF1-specific antibodies are available, binding interactions with EPHA2, BAG4, or ARF1 can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

EPHA2, BAG4, or ARF1 can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case EPHA2, BAG4, or ARF1 or antigenic subsequence thereof).

Immunoassays also often use a labeling agent to specifically bind to and label the complex formed by the antibody and antigen. The labeling agent may itself be one of the moieties comprising the antibody/antigen complex. Thus, the labeling agent may be a labeled EPHA2, BAG4, or ARF1 polypeptide or a labeled anti-EPHA2, BAG4, or ARF1 antibody. Alternatively, the labeling agent may be a third moiety, such as a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the labeling agent. These proteins exhibit a strong non-immunogenic reactivity with immunoglobulin constant regions from a variety of species (see, e.g., Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Commonly used assays include noncompetitive assays, e.g., sandwich assays, and competitive assays. In competitive assays, the amount of EPHA2, BAG4, or ARF1 present in the sample is measured indirectly by measuring the amount of a known, added (exogenous) EPHA2, BAG4, or ARF1 displaced (competed away) from an anti-EPHA2, BAG4, or ARF1 antibody by the unknown EPHA2, BAG4, or ARF1 present in a sample. Commonly used assay formats include immunoblots, which are used to detect and quantify the presence of protein in a sample. Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels, enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize EPHA2, BAG4, or ARF1, or secondary antibodies that recognize anti-EPHA2, BAG4, or ARF1.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple calorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Cross-Reactivity Determinations

Immunoassays in the competitive binding format can also be used for cross-reactivity determinations. For example, a protein at least partially encoded by SEQ NO:1, 3, or 5; can be immobilized to a solid support. Proteins (e.g., EPHA2, BAG4, or ARF1 protein variants or homologs) are added to the assay that compete for binding of the antisera to the immobilized antigen. The ability of the added proteins to compete for binding of the antisera to the immobilized protein is compared to the ability of EPHA2, BAG4, or ARF1 encoded by SEQ ID NO:1, 3, or 5 to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of EPHA2, BAG4, or ARF1, to the immunogen protein (i.e., the EPHA2, BAG4, or ARF1 of SEQ ID NO:2, 4, or 6). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the antigenic protein that is required to inhibit 50% of binding, then the second protein is said to specifically bind to the polyclonal antibodies generated to a EPHA2, BAG4, or ARF1 immunogen.

Detection of Activity

As appreciated by one of skill in the art, EPHA2, BAG4, or ARF1 activity can be detected to evaluate expression levels or for identifying modulators of activity. The activity can be assessed using a variety of in vitro and in vivo assays to determine functional, chemical, and physical effects, e.g., measuring ligand binding, measuring second messengers (e.g., cAMP, cGMP, IP3, DAG, or Ca²⁺), measuring phosphorylation levels, measuring apoptosis, measuring transcription levels, measuring indicators of transformation, e.g., growth in soft agar, change in cell phenotype, change in the mitotic index, and the like. For example, EPHA2 is a tyrosine kinase. Activity can therefore be determined by measuring phosphorylation or can be determined by measuring other endpoints, e.g., cell growth, growth in soft agar, and the like. Similarly, BAG4 activity can be detected by examining its ability to bind to TNFR1, or by evaluating apoptosis levels. ARF1 activity can also be determined be evaluating its activity as a small guanine nucleotide-binding protein, by its ability to activate phospholipase D or by evaluating a downstream effect of the protein, e.g., cell growth.

Screening assays of the invention are used to identify modulators that can be used as therapeutic agents, e.g., antibodies to EPHA2, BAG4, or ARF1 and antagonists of EPHA2, BAG4, or ARF1 activity.

The EPHA2, BAG4, or ARF1 for the assay is often selected from a polypeptide having a sequence of SEQ ID NO:2, 4, or 6, or conservatively modified variants thereof. Alternatively, the EPHA2, BAG4, or ARF1 will be derived from a eukaryote and include an amino acid subsequence having amino acid sequence identity to SEQ ID NO:2, 4, or 6. Generally, the amino acid sequence identity will be at least 70%, optionally at least 80%, or 90-95%. The EPHA2, BAG4, or ARF1 typically comprises at least 10 contiguous amino acids, often at least 20, 50, 100, 200, or 300 contiguous amino acids of SEQ ID NO:2, 4, or 6. Optionally, the polypeptide of the assays will comprise or consist of a domain of EPHA2, BAG4, or ARF1, such as a ligand binding domain, subunit association domain, active site, and the like. Either a EPHA2, BAG4, or ARF1 or a domain thereof can be covalently linked to a heterologous protein to create a chimeric protein used in the assays described herein.

Modulators of EPHA2, BAG4, or ARF1 activity are tested using EPHA2, BAG4, or ARF1 polypeptides as described above, either recombinant or naturally occurring. The protein can be isolated, expressed in a cell, expressed in a membrane derived from a cell, expressed in tissue or in an animal, either recombinant or naturally occurring. For example, transformed cells or membranes can be used. Modulation is tested using one of the in vitro or in vivo assays described herein. Activity can can also be examined in vitro with soluble or solid state reactions, using a chimeric molecule such as a ligand binding domain of a receptor covalently linked to a heterologous signal transduction domain. Furthermore, ligand-binding domains of the protein of interest can be used in vitro in soluble or solid state reactions to assay for ligand binding.

Ligand binding to EPHA2, BAG4, or ARF1, a domain, or a chimeric protein can be tested in a number of formats. Binding can be performed in solution, in a bilayer membrane, attached to a solid phase, in a lipid monolayer, or in vesicles. Often, in an assay of the invention, the binding of a candidate ligand to EPHA2, BAG4, or ARF1 is measured in the presence of a known ligand. Often, competitive assays that measure the ability of a compound to compete with binding of a known ligand to the receptor are used. Binding can be tested by measuring, e.g., changes in spectroscopic characteristics (e.g., fluorescence, absorbance, refractive index), hydrodynamic (e.g., shape) changes, or changes in chromatographic or solubility properties.

In another embodiment, transcription levels can be measured to assess the effects of a test compound on EPHA2, BAG4, or ARF1. A host cell expressing EPHA2, BAG4, or ARF1 is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter genes may be used as described in U.S. Pat. No. 5,436,128, herein incorporated by reference. The reporter genes can be, e.g., chloramphenicol acetyltransferase, firefly luciferase, bacterial luciferase, β-galactosidase and alkaline phosphatase. (1997)).

The amount of transcription is then compared to the amount of transcription in either the same cell in the absence of the test compound. A substantially identical cell may be derived from the same cells from which the recombinant cell was prepared but which had not been modified by introduction of heterologous DNA. Any difference in the amount of transcription indicates that the test compound has in some manner altered the activity of the protein of interest.

In assays to identify EPHA2, BAG4, or ARF1 inhibitors, samples that are treated with a potential inhibitor are compared to control samples to determine the extent of modulation. Control samples (untreated with candidate inhibitors) are assigned a relative activity value of 100. Inhibition of EPHA2, BAG4, or ARF1 is achieved when the activity value relative to the control is about 90%, optionally 50%, optionally 25-0%.

Candidate Compounds

The compounds tested as inhibitors of EPHA2, BAG4, or ARF1 can be any small chemical compound, or a biological entity, e.g., a macromolecule such as a protein, sugar, nucleic acid or lipid. Alternatively, modulators can be genetically altered versions of EPHA2, BAG4, or ARF1. Typically, test compounds will be small chemical molecules and peptides or antibodies.

Essentially any chemical compound can be used as a potential modulator or ligand in the assays of the invention. Most often, compounds can be dissolved in aqueous or organic (especially DMSO-based) solutions. The assays are designed to screen large chemical libraries by automating the assay steps, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Russell & Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Solid State and Soluble High Throughput Assays

In one embodiment the invention provides soluble assays using molecules such as a domain, e.g., a ligand binding domain, an active site, a subunit association region, etc.; a domain that is covalently linked to a heterologous protein to create a chimeric molecule; a EPHA2, BAG4, or ARF1; or a cell or tissue expressing a EPHA2, BAG4, or ARF1, either naturally occurring or recombinant. In another embodiment, the invention provides solid phase based in vitro assays in a high throughput format, where the domain, chimeric molecule, EPHA2, BAG4, or ARF1, or cell or tissue expressing EPHA2, BAG4, or ARF1 is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-1500 different compounds. It is possible to assay several different plates per day; assay screens for up to about 6,000-20,000 different compounds is possible using the integrated systems of the invention.

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (e.g., the signal transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and are appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Computer-Based Assays

Yet another assay for compounds that modulate EPHA2, BAG4, or ARF1 activity involves computer assisted drug design, in which a computer system is used to generate a three-dimensional structure of EPHA2, BAG4, or ARF1 based on the structural information encoded by the amino acid sequence. The input amino acid sequence interacts directly and actively with a pre-established algorithm in a computer program to yield secondary, tertiary, and quaternary structural models of the protein. The models of the protein structure are then examined, for example, to identify the regions that have the ability to bind ligands. These regions are then used to identify various compounds that inhibit ligand-receptor binding.

The three-dimensional structural model of the protein is generated by entering protein amino acid sequences of at least 10 amino acid residues or corresponding nucleic acid sequences encoding a EPHA2, BAG4, or ARF1 polypeptide into the computer system. The amino acid sequence may comprise SEQ ID NO: 2, 4, or 8. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of EPHA2, BAG4, or ARF1 to identify ligands that bind to the EPHA2, BAG4, or ARF1. Binding affinity between the protein and ligands is determined using energy terms to determine which ligands have an enhanced probability of binding to the protein.

Expression Assays

Certain screening methods involve screening for a compound that modulates the expression of EPHA2, BAG4, or ARF1. Such methods generally involve conducting cell-based assays in which test compounds are contacted with one or more cells expressing a EPHA2, BAG4, or ARF1 and then detecting a decrease in expression (either transcript or translation product). Such assays are often performed with cells that overexpress EPHA2, BAG4, or ARF1.

Expression can be detected in a number of different ways. As described herein, the expression levels of the protein in a cell can be determined by probing the mRNA expressed in a cell with a probe that specifically hybridizes with a EPHA2, BAG4, or ARF1 transcript (or complementary nucleic acid derived therefrom). Alternatively, protein can be detected using immunological methods in which a cell lysate is probed with antibodies that specifically bind to the protein.

Other cell-based assays are reporter assays conducted with cells that do not express the protein. Often, these assays are conducted with a heterologous nucleic acid construct that includes a promoter that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, CAT (chloramphenicol acetyl transferase), luciferase, β-galactosidase and alkaline phosphatase.
n these assays, cells harboring the reporter construct are contacted with a test compound. A test compound that inhibits the activity of the promoter, e.g., by binding to it or triggering a cascade that produces a molecule that decreases the promoter-induced expression of the detectable reporter can be detected by comparison to control cells that have not been treated with the inhibitor. Certain other reporter assays are conducted with cells that harbor a heterologous construct that includes a transcriptional control element that activates expression of EPHA2, BAG4, or ARF1 and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

In another embodiment, EPHA2, BAG4, or ARF1 are used to generate animal models of breast cancer. For example, a transgenic animals can be generated that overexpresses EPHA2, BAG4, or ARF1. Depending on the desired expression level, promoters of various strengths can be employed to express the transgene. Also, the number of copies of the integrated transgene can be determined and compared for a determination of the expression level of the transgene. Animals generated by such methods can be used for screening for inhibitors to treat breast cancer.

Disease Treatment and Diagnosis/Prognosis

EPHA2, BAG4, or ARF1 nucleic acid and polypeptide sequences can be used for diagnosis or prognosis of breast cancer in a patient. For example, the sequence, level, or activity of EPHA2, BAG4, or ARF1 in a patient can be determined, wherein an alteration, e.g., an increase in the level of expression or activity of t EPHA2, BAG4, or ARF1, or the detection of an increase in copy number or mutations in the EPHA2, BAG4, or ARF1, indicates the presence or the likelihood of breast cancer.

Often, such methods will be used in conjunction with additional diagnostic methods, e.g., detection of other breast cancer indicators, e.g., cell morphology, HER2/neu expression, and the like. In other embodiments, a tissue sample known to contain cancerous cells, e.g., from a tumor, will be analyzed for EPHA2, BAG4, or ARF1 levels to determine information about the cancer, e.g., the efficacy of certain treatments, the survival expectancy

In some embodiments, the level of EPHA2, BAG4, or ARF1 can be used to determine the prognosis of a patient with breast cancer. For example, if cancer is detected using a technique other than by detecting EPHA2, BAG4, or ARF1, e.g., tissue biopsy, then the presence or absence of EPHA2, BAG4, or ARF1 can be used to determine the prognosis for the patient, i.e., an elevated level of EPHA2, BAG4, or ARF1 will typically indicate a reduced survival expectancy in the patient compared to in a patient with cancer but with a normal level of EPHA2, BAG4, or ARF1. As used herein, “survival expectancy” refers to a prediction regarding the severity, duration, or progress of a disease, condition, or any symptom thereof. In a preferred embodiment, an increased level, a diagnostic presence, or a quantified level, of EPHA2, BAG4, or ARF1 is statistically correlated with the observed progress of a disease, condition, or symptom in a large number of patients, thereby providing a database wherefrom a statistically-based prognosis can be made. For example, in a particular type of patient, a human of a particular age, gender, medical condition, medical history, etc., a detection of a level of EPHA2, BAG4, or ARF1 that is, e.g., 2 fold higher than a control level may indicate, e.g., a 10% reduced survival expectancy in the human compared to in a similar human with a normal level of EPHA2, BAG4, or ARF1, based on a previous study of the level of EPHA2, BAG4, or ARF1 in a large number of similar patients whose disease progression was observed and recorded.

The methods of the present invention can be used to determine the optimal course of treatment in a patient with breast cancer. For example, the presence of an elevated level of EPHA2, BAG4, or ARF1 can indicate a reduced survival expectancy of a patient with cancer, thereby indicating a more aggressive treatment for the patient In addition, a correlation can be readily established between levels of EPHA2, BAG4, or ARF1, or the presence or absence of a diagnostic presence of EPHA2, BAG4, or ARF1, and the relative efficacy of one or another anti-cancer agent. Such analyses can be performed, e.g., retrospectively, i.e., by detecting EPHA2, BAG4, or ARF1 levels in samples taken previously from patients that have subsequently undergone one or more types of anti-cancer therapy, and correlating the EPHA2, BAG4, or ARF1 levels with the known efficacy of the treatment.

Administration of Pharmaceutical and Vaccine Compositions

Inhibitors of EPHA2, BAG4, or ARF1 can be administered to a patient for the treatment of breast cancer. As described in detail below, the inhibitors are administered in any suitable manner, optionally with pharmaceutically acceptable carriers.

The identified inhibitors can be administered to a patient at therapeutically effective doses to prevent, treat, or control breast cancer. The compounds are administered to a patient in an amount sufficient to elicit an effective protective or therapeutic response in the patient. An effective therapeutic response is a response that at least partially arrests or slows the symptoms or complications of the disease. An amount adequate to accomplish this is defined as “therapeutically effective dose.” The dose will be determined by the efficacy of the particular EPHA2, BAG4, or ARF1 inhibitors employed and the condition of the subject, as well as the body weight or surface area of the area to be treated. The size of the dose also will be determined by the existence, nature, and extent of any adverse effects that accompany the administration of a particular compound or vector in a particular subject.

Toxicity and therapeutic efficacy of such compounds can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a modulator is from about 1 ng/kg to 10 mg/kg for a typical subject.

Pharmaceutical compositions for use in the present invention can be formulated by standard techniques using one or more physiologically acceptable carriers or excipients. The compounds and their physiologically acceptable salts and solvates can be formulated for administration by any suitable route, including via inhalation, topically, nasally, orally, parenterally (e.g., intravenously, intraperitoneally, intravesically or intrathecally) or rectally.

For oral administration, the pharmaceutical compositions can take the form of, for example, tablets or capsules prepared by conventional means with pharmaceutically acceptable excipients, including binding agents, for example, pregelatinised maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. Tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use.

The compounds can also be formulated in rectal compositions, for example, suppositories or retention enemas, for example, containing conventional suppository bases, for example, cocoa butter or other glycerides.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

Inhibitors of Gene Expression

In one aspect of the present invention, EPHA2, BAG4, or ARF1 inhibitors can also comprise nucleic acid molecules that inhibit expression of EPHA2, BAG4, or ARF1. Conventional viral and non-viral based gene transfer methods can be used to introduce nucleic acids encoding engineered EPHA2, BAG4, or ARF1 polypeptides in mammalian cells or target tissues, or alternatively, nucleic acids e.g., inhibitors of EPHA2, BAG4, or ARF1 activity, such as siRNAs or anti-sense RNAs. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

In some embodiments, small interfering RNAs are administered. In mammalian cells, introduction of long dsRNA (>30 nt) often initiates a potent antiviral response, exemplified by nonspecific inhibition of protein synthesis and RNA degradation. The phenomenon of RNA interference is described and discussed, e.g., in Bass, Nature 411:428-29 (2001); Elbahir et al., Nature 411:494-98 (2001); and Fire et al., Nature 391:806-11 (1998), where methods of making interfering RNA also are discussed. The siRNAs based upon the EPHA2, BAG4, or ARF1 sequences disclosed herein are less than 100 base pairs, typically 30 bps or shorter, and are made by approaches known in the art. Exemplary siRNAs according to the invention could have up to 29 bps, 25 bps, 22 bps, 21 bps, 20 bps, 15 bps, 10 bps, 5 bps or any integer thereabout or therebetween.

Non-Viral Delivery Methods

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Viral Delivery Methods

The use of RNA or DNA viral based systems for the delivery of inhibitors of EPHA2, BAG4, or ARF1 are known in the art. Conventional viral based systems for the delivery of EPHA2, BAG4, or ARF1 nucleic acid inhibitors can include retroviral, lentivirus, adenoviral, adeno-associated and herpes simplex virus vectors for gene transfer.

In many gene therapy applications, it is desirable that the gene therapy vector be delivered with a high degree of specificity to a particular tissue type, e.g., a joint or the bowel. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient.

Ex vivo cell transfection for diagnostics, research, or for gene therapy (e.g., via re-infusion of the transfected cells into the host organism) is well known to those of skill in the art. In some embodiments, cells are isolated from the subject organism, transfected with EPHA2, BAG4, or ARF1 inhibitor nucleic acids and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Vectors (e.g., retroviruses, adenoviruses, liposomes, etc.) containing therapeutic nucleic acids can also be administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington 's Pharmaceutical Sciences, 17th ed., 1989).

In some embodiments, EPHA2, BAG4, and ARF1 polypeptides and polynucleotides can also be administered as vaccine compositions to stimulate an immune response, typically a cellular (CTL and/or HTL) response. Such vaccine compositions can include, e.g., lipidated peptides (see, e.g., Vitiello, A. et al., J. Clin. Invest. 95:341 (1995)), peptide compositions encapsulated in poly(DL-lactide-co-glycolide) (“PLG”) microspheres (see, e.g., Eldridge, et al., Molec. Immunol. 28:287-294, (1991); Alonso et al., Vaccine 12:299-306 (1994); Jones et al., Vaccine 13:675-681 (1995)), peptide compositions contained in immune stimulating complexes (ISCOMS) (see, e.g., Takahashi et al., Nature 344:873-875 (1990); Hu et al., Clin Exp Immunol. 113:235-243 (1998)), multiple antigen peptide systems (MAPs) (see, e.g., Tam, Proc. Natl. Acad. Sci. U.S.A. 85:5409-5413 (1988); Tam, J. Immunol. Methods 196:17-32 (1996)), peptides formulated as multivalent peptides; peptides for use in ballistic delivery systems, typically crystallized peptides, viral delivery vectors (Perkus, et al., In: Concepts in vaccine development (Kaufmann, ed., p. 379, 1996); Chakrabarti, et al., Nature 320:535 (1986); Hu et al., Nature 320:537 (1986); Kieny, et al., AIDS Bio/Technology 4:790 (1986); Top et al., J. Infect. Dis. 124:148 (1971); Chanda et al., Virology 175:535 (1990)), particles of viral or synthetic origin (see, e.g., Kofler et al., J. Immunol. Methods. 192:25 (1996); Eldridge et al., Sem. Hematol. 30:16 (1993); Falo et al., Nature Med. 7:649 (1995)), adjuvants (Warren et al., Annu. Rev. Immunol. 4:369 (1986); Gupta et al., Vaccine 11:293(1993)), liposomes (Reddy et al., J. Immunol. 148:1585(1992); Rock, Immunol. Today 17:131 (1996)), or, naked or particle absorbed cDNA (Ulmer, et al., Science 259:1745 (1993); Robinson et al., Vaccine 11:957 (1993); Shiver et al., In: Concepts in vaccine development (Kaufmann, ed., p. 423, 1996); Cease & Berzofsky, Annu. Rev. Immunol. 12:923 (1994) and Eldridge et al., Sem. Hematol. 30:16 (1993)). Toxin-targeted delivery technologies, also known as receptor mediated targeting, such as those of Avant Immunotherapeutics, Inc. (Needham, Mass.) may also be used.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, breast cancer-specific nucleic acids or antibodies, hybridization probes and/or primers, antisense polynucleotides, siRNAs, ribozymes, dominant negative breast cancer polypeptides or polynucleotides, small molecules inhibitors of breast cancer-associated sequences etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of breast cancer-associated sequences. Such kits can be prepared from readily available materials and reagents. For example, such kits can comprise one or more of the following materials: a breast cancer-associated polypeptide or polynucleotide, reaction tubes, and instructions for testing breast cancer-associated activity. Optionally, the kit contains biologically active breast cancer protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

### EXAMPLES

We have assessed gene amplification in over 150 primary breast tumors and 50 breast cancer cell lines using array CGH In addition, we have assessed gene expression using Affymetrix U133A expression arrays in the cell lines. These studies have identified several genes including EPHA2, BAG4 and ARF1 that are recurrently amplified and over expressed when amplified.

Array CGH and Genome Analysis. Array CGH has proved to be a powerful tool for identification of regions of recurrent genomic abnormality. The principle advantages of array CGH are that it maps changes in copy number throughout a complex genome onto a normal reference genome so the aberrations can be easily related to existing physical maps, genes, and genomic DNA sequence, and it employs genomic DNA so that cell culture is not required. The resolution with which genome copy number can be detected and mapped is defined by the genomic spacing of the clones used to form the array. Arrays now in use are comprised of 2500 BACs distributed at ˜1 MB intervals over the genome plus ˜2200 BACs selected to target genes involved in receptor tyrosine kinase signaling or regions of recurrent abnormalities identified in earlier studies. Furthermore, array CGH allows quantitative assessment of genome dosage from one copy per test genome to hundreds of copies per genome.

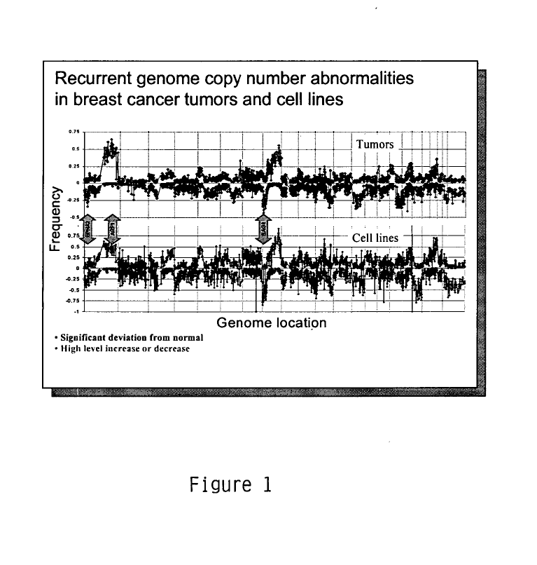

To date, we have analyzed over 150 primary breast tumors and 50 breast cancer cell lines using. Regions of recurrent abnormality are summarized in FIG. 1. Recurrent abnormalities can be assessed computationally for gene content using Genome Cryptographer (a sequence annotation tool developed by us for this purpose), private databases, and the UC Santa Cruz web site at http://genome.ucsc.edu. In general, the regions of abnormality in the cell lines are similar to those in the primary tumors indicating that functional assessment of aberrations in the cell lines will be directly relevant to the primary tumors.

Gene amplification is a well-established mechanism of increasing the expression of oncogenes, the archetypal gene being ERBB2. However, not all amplified genes are over expressed. In fact recent estimates suggest that less than half of all highly amplified genes are over expressed. Accordingly, we have assessed gene expression in the breast cancer cell lines using Affymetrix U133A arrays, analysis of gene copy number using array CGH and protein expression profiling on a panel of 60 human breast cancer cell lines has enabled us to identify over 200 amplified genes whose expression is strongly correlated with genome copy number. We have chosen two of these, ARF1 and BAG4, as clinical therapeutic targets for the treatment of breast cancer because they are frequently amplified in primary breast tumors and because their levels of amplification are strongly correlated with their levels of expression (See Table 1).

We also assessed expression of several genes associated with receptor tyrosine kinase signaling at the protein level. The receptor tyrosine kinase, EPHA2, is particularly interesting because its expression is almost perfectly anticorrelated with the expression of ERBB3 (see FIG. 4 below). Thus, agents targeting EPHA2 may be useful in patients that are not candidates for treatment with Herceptin or other agents that target tumors expressing ERBB3.

TABLE 1
Description of genes chosen for study. ERBB2 is included for
comparison to ARF1 and BAG4, as it is the classic example of
gene amplification and over-expression in cancer. The percentage
of cells and tumors exhibiting amplification reflects those samples
with at least two-fold amplification.

| | | | % Cell | | |
|---|---|---|---|---|---|
| | | | lines | % Tumors | |
| | | Pearsons | with | with | |
| | | Correla- | Ampli- | Ampli- | |
| Gene | Chr | tion | fication | fication | Description |
| ERBB2 | 17q12 | 0.91 | 26 | 14 | Receptor tyrosine |
| | | | | | kinase |
| ARG1 | 1q42 | 0.75 | 38 | 14 | ADP-ribosylation |
| | | | | | factor |
| BAG4 | 8p12 | 0.85 | 28 | 20 | Silencer of Death |
| | | | | | Domains |
| EPHA2 | 1p36.13 | — | — | — | Receptor tyrosine |
| | | | | | kinase |

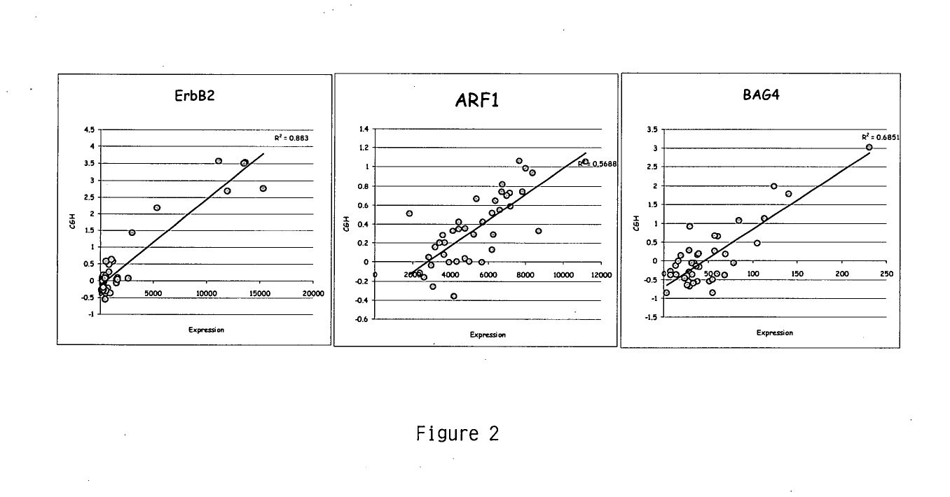

BAG4 and ARF1. These genes were selected based on their strong correlation between gene amplification and expression. FIG. 2 shows gene copy number plotted against gene expression levels for these genes and for the model example, ERBB2. The data clearly show the increased copy number leads to gene over-expression in a manner comparable to that of ERBB2.

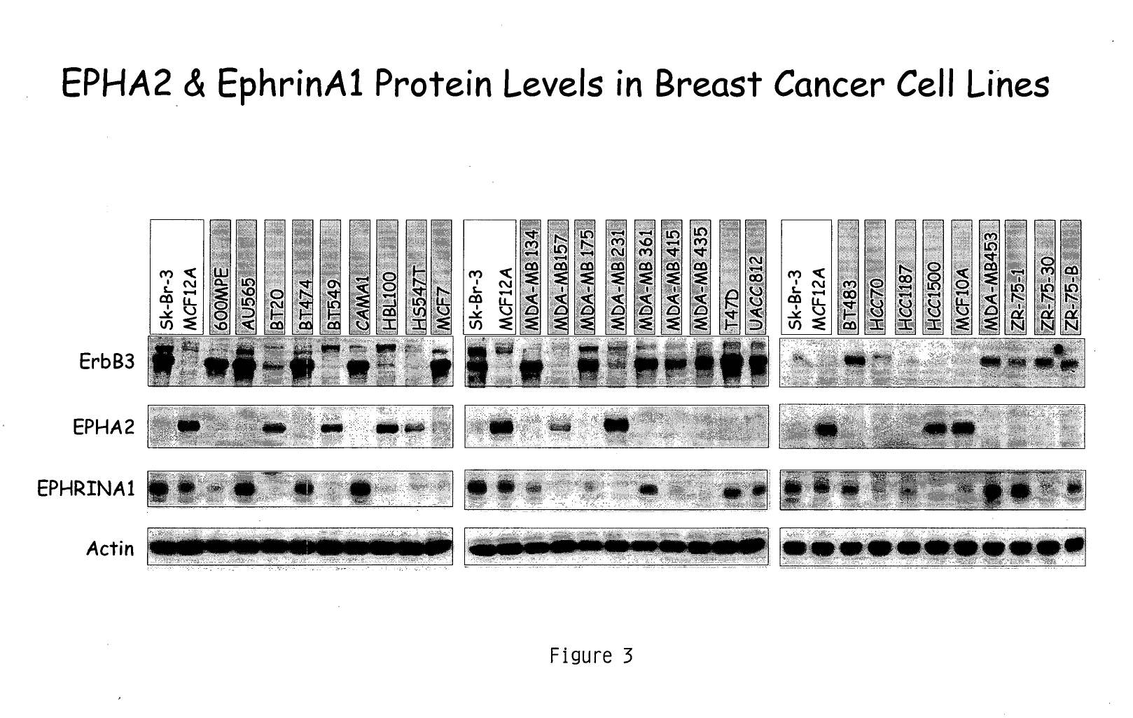

EPHA2. Protein expression profiling of the breast cell lines has revealed a striking inverse relationship between the expression of two receptor tyrosine kinases EPHA2 and ERBB3 (FIG. 3). Western blots of whole cell lysates from human breast cancer cell lines revealed an inverse relationship between ERBB3 and EPHA2 expression across all samples. EPHA2 is found expressed in the more aggressive cell lines, which constitutes approximately 30% of samples analysed. Ligand, e.g., ephrin, stimulation of EPHA2 leads to receptor phosphorylation, and down regulation. In three-dimensional cultures we have observed that this reverts the invasive, malignant phenotype of EPHA2 positive cells to a normal phenotype.

Cell System that Constitutively Over-Expresses the Target Gene for the Analysis of Modulators

This example shows how cell lines to identify inhibitors may be generated. MCF10A cell lines that constitutively over express the target genes are are established to assay for modulators of EPHA2, ARF1, and BAG4. Expression vectors encoding EPHA2, ARF1 and BAG4 will be introduced into genomically near-normal MCF10A breast epithelial cells using retroviral infection and standard selection protocols. The normal breast cell line, MCF10A, cam be transformed by oncogenes such as ERBB2 (MCF10A-NT), forming colonies in soft agar. MCF 10A-NT cells will be used as a positive controls. Negative controls are cells infected with the backbone vector selected under the same conditions.

Biological responses (e.g., apoptosis, motility, morphology, cell number, viability, mitotic index, and celly cycle distribution) can be measured in EPHA2, ARF1, or BAG4-transformed cells. Response will be assessed using a flow cytometer equipeed with a 96-well reader and a Cellomics HCS ArrayScan system for high content imaging. The BD cytometer, allows automated plate analysis and output to a standard database file with user defined keywords and sample identification. It will be used to measure DNA distributions and an apoptotic index during treatment. For this assay, cells will be fixed in 70% ethanol, treated with RNase, stained with propidium iodide (PI), and placed in 96 well trays. The PI fluorescence distributions will be analysed to determine the fractions of cells in the G1-, S-, and G2M phases of the cell cycle and for the fraction of “sub diploid” cells as an apoptotic index.

The Arrayscan system is an automated imaging instrument that scans through the bottom of clear bottom multi well plates, focuses on a field of cells, and acquires images at each selected color channel. The ArrayScan software identifies and measures individual features and structures within each cell in a field of cells, so that up to hundreds of cell samples can be analysed in parallel. The software then tabulates and presents the results in user defined formats, The systcan will be used to assess cell number mitotic index, motility and apoptosis.

Mitotic index. Cells undergoing cell division within a population will be identified using the ArrayScan II based on microtubule spindle formation and chromosome condensation using the Cellomics Mitotic Indext HitKit™. Following compound treatment; cells growing in standard high density plates will be fixed, permeabilized, and immunofluorescently labelled using an antibody specific for aphosphrylated epitope of a core histone protein.

Cell Motility. Cell motility will be assessed using the ArrayScan II by directly measuring the size of tracks generated by migrating cells using the Cellomics Mitotic Indext HitKit™. The assay is performed on live cells plated on a lawn of microscopic fluorescent beads. As cells move across the lawn, they leave clear tracks behind. The track area is measured as an estimate of the rate of cell movement.

Proliferation and Apoptosis. Increases in proliferation and/or decreases in apoptosis (increased survival) are common mechanisms of oncogenesis. Apoptotic cells will be detected based on nuclear morphology, mitochondrial mass and/or membrane potential, and f-actin content following staining with rte Cellomics Multiparameter Apoptosis 1 HitKit™. Nuclear morphology (i.e., condensation or fragmentation) will be measured after staining with Hoechst 33258. Mitochondrial membrane potential and mitochondrial mass will be measured after staining with MitoTracker® Red. F actin will be measured after staining with an Alexa Fiuor® 488 conjugate of phalloidin (Ax488-ph).

Flow cytometry and time lapse videomicroscopy also will be used to assess the effects of infection with EPHA2, BAG4 end ARF1. Proliferation will be measured relative to control cells using propidium iodide (P1) staining to assess the cell cycle distribution (GO/G1, S, G2/M) of the cell population. 5 bromodeoxyuridine labelling will be used to assess mitotic index. PI staining will also yield data on apoptosis, as measured by the presence of a sub-G1 peak, a characteristic of apoptotic cells Cells will also be monitored over the course of 1-4 days by CCD based digital imaging every 5 10 minutes. Onset of apoptosis will be scored by the appearance of plasma membrane blebbing, and apoptotic cell death will be scored when the cell have completely deteached from the surface of the culture dish. Proliferation and motility kinetics will be determined by measuring inter-mitotic time and total cell number (adjusted for loss of apoptotic cells).

Soft agar colony formation assay. Loss of anchorage dependent growth is a result of oncogene activation. The effects of modulators can also be tested on infected MCF10A by analyzing the cells for anchorage independent growth properties based on their ability to form colonies its soft agar using standard techniques. Briefly, cells will be mixed with agar and culture media, plated onto base agar, and incubated for 10-14 days. Plates will be stained with Crystal Violet and colonies counted using a dissecting microscope.

Candidate modulators can further be identified by selecting those compounds that inhibit EPHA2, BAG4, or ARF1 in a cellular assay and validating the compound in vivo using a system in which the inhibitor is applied to tumor xenografts in which the EPHA2, BAG4, or ARF1 gene is highly amplified and over-expressed. In this approach, immune deficient mice (nu/nu and scid) carrying human tumor breast cancer xenografts will be used for pre clinical evaluation of the tumorigenicity of target gene inhibitors. Tumor growth will be measured over 25 days, at which point the candidate compound or placebo (PBS control) will be administered. Tumor growth will be followed for an additional 15 day. Tumors will then be removed and evaluated by immunohistochemical and biochemical analysis.

The above examples are provided by way of illustration only and not by way of limitation. Those of skill in the art will readily recognize a variety of noncritical parameters that could be changed or modified to yield essentially similar results.

All publications and patent applications cited in this specification are herein incorporated by reference as if each individual publication or patent application were specifically and individually indicated to be incorporated by reference.

TABLE OF SEQUENCES

SEQ ID NO:1 human BAG4 nucleic acid sequence

| | |
|---|---|
| 1 | aggtaagagg aaactccatt ggataaatgg cgaggaaacg tatactccct cttaaggaac |
| 61 | acggtgtctt ccttcgtctc cgggttcccg agaccccaga gtcactgacc tccgtccctc |
| 121 | agctttcggg gttcggcagc agaaggggcg ggcccgggcc tgggattggc tggcgtcgtc |
| 181 | cgaccccctt cgctgctctc cattcgcaat cgcccgcggg cgcctgcgcg atgggtcggc |
| 241 | cgtggggagc ggggcgggaa gcgcttcagg gcagcggatc ccatgtcggc cctgaggcgc |
| 301 | tcgggctacg gccccagtga cggtccgtcc tacggccgct actacgggcc tgggggtgga |
| 361 | gatgtgccgg tacacccacc tccaccctta tatcctcttc gccctgaacc tccccagcct |
| 421 | cccatttcct ggcgggtgcg cgggggcggc ccggcggaga ccacctggct gggagaaggc |
| 481 | ggaggaggcg atggctacta tccctcggga ggcgcctggc cagagcctgg tcgagccgga |
| 541 | ggaagccacc aggagcagcc accatatcct agctacaatt ctaactattg gaattctact |
| 601 | gcgagatcta gggctcctta cccaagtaca tatcctgtaa gaccagaatt gcaaggccag |
| 661 | agtttgaatt cttatacaaa tggagcgtat ggtccaacat accccccagg ccctggggca |
| 721 | aatactgcct catactcagg ggcttattat gcacctggtt atactcagac cagttactcc |
| 781 | acagaagttc caagtactta ccgttcatct ggcaacagcc caactccagt ctctcgttgg |
| 841 | atctatcccc agcaggactg tcagactgaa gcaccccctc ttagggggca ggttccagga |
| 901 | tatccgcctt cacagaaccc tggaatgacc ctgccccatt atccttatgg agatggtaat |
| 961 | cgtagtgttc cacaatcagg accgactgta cgaccacaag aagatgcgtg ggcttctcct |
| 1021 | ggtgcttatg gaatgggtgg ccgttatccc tggccttcat cagcgccctc agcaccaccc |
| 1081 | ggcaatctct acatgactga aagtacttca ccatggccta gcagtggctc tccccagtca |
| 1141 | cccccttcac ccccagtcca gcagcccaag gattcttcat acccctatag ccaatcagat |
| 1201 | caaagcatga accggcacaa ctttccttgc agtgtccatc agtacgaatc ctcggggaca |
| 1261 | gtgaacaatg atgattcaga tcttttggat tcccaagtcc agtatagtgc tgagcctcag |
| 1321 | ctgtatggta atgccaccag tgaccatccc aacaatcaag atcaaagtag cagtcttcct |
| 1381 | gaagaatgtg taccttcaga tgaaagtact cctccgagta ttaaaaaaat catacatgtg |
| 1441 | ctggagaagg tccagtatct tgaacaagaa gtagaagaat ttgtaggaaa aaagacagac |
| 1501 | aaagcatact ggcttctgga agaaatgcta accaaggaac ttttggaact ggattcagtt |
| 1561 | gaaactgggg gccaggactc tgtacggcag gccagaaaag aggctgtttg taagattcag |
| 1621 | gccatactgg aaaaattaga aaaaaaagga ttatgaaagg atttagaaca aagtggaagc |
| 1681 | ctgttactaa cttgaccaaa gaacacttga tttggttaat taccctcttt ttgaaatgcc |
| 1741 | tgttgatgac aagaagcaat acattccagc tttcctttga ttttatactt gaaaaactgg |
| 1801 | caaaggaatg gaagaatatt ttagtcatga gttgttttca gttttcagac gaatgaatgt |
| 1861 | aataggaaac tatggagtta ccaatattgc caagtagact cactccttaa aaaatttatg |
| 1921 | gatatctaca agctgcttct taccagcagg agggaaacac acttcacaca acaggcttat |
| 1981 | cagaaaccta ccagatgaaa ctggatataa tctgagacaa acaggatgtg tttttttaaa |
| 2041 | catctggata tcttgtcaca tttttgtaca ttgtgactgc tttcaacata tacttcatgt |
| 2101 | gtaattatag cttagacttt agccttcttg gacttctgtt ttgttttgtt atttgcagtt |
| 2161 | tacaaatata gtattattct ct |

SEQ ID NO: 2 human BAG4 polypeptide sequence
MSALRRSGYGPSDGPSYGRYYGPGGGDVPVHPPPPLYPLRPEPP
QPPISWRVRGGGPAETTWLGEGGGGDGYYPSGGAWPEPGRAGGSHQEQPPYPSYNSNY
WNSTARSRAPYPSTYPVRPELQGQSLNSYTNGAYGPTYPPGPGANTASYSGAYYAPGY
TQTSYSTEVPSTYRSSGNSPTPVSRWIYPQQDCQTEAPPLRGQVPGYPPSQNPGMTLP
HYPYGDGNRSVPQSGPTVRPQEDAWASPGAYGMGGRYPWPSSAPSAPPGNLYMTESTS
PWPSSGSPQSPPSPPVQQPKDSSYPYSQSDQSMNRHNFPCSVHQYESSGTVNNDDSDL
LDSQVQYSAEPQLYGNATSDHPNNQDQSSSLPEECVPSDESTPPSIKKIIHVLEKVQY
LEQEVEEFVGKKTDKAYWLLEEMLTKELLELDSVETGGQDSVRQARKEAVCKIQAILE
KLEKKGL
SEQ ID NO: 3 human ARF1 nucleic acid sequence

| | |
|---|---|
| 1 | gcaaaaccaa cgcctggctc ggagcagcag cctctgaggt gtccctggcc agtgtccttc |
| 61 | cacctgtcca caagcatggg gaacatcttc gccaacctct tcaagggcct ttttggcaaa |
| 121 | aaagaaatgc gcatcctcat ggtgggcctg gatgctgcag ggaagaccac gatcctctac |
| 181 | aagcttaagc tgggtgagat cgtgaccacc attcccacca taggcttcaa cgtggaaacc |
| 241 | gtggagtaca agaacatcag cttcactgtg tgggacgtgg gtggccagga caagatccgg |
| 301 | cccctgtggc gccactactt ccagaacaca caaggcctga tcttcgtggt ggacagcaat |
| 361 | gacagagagc gtgtgaacga ggcccgtgag gagctcatga ggatgctggc cgaggacgag |
| 421 | ctccgggatg ctgtcctcct ggtgttcgcc aacaagcagg acctccccaa cgccatgaat |
| 481 | gcggccgaga tcacagacaa gctggggctg cactcactac gccacaggaa ctggtacatt |
| 541 | caggccacct gcgccaccag cggcgacggg ctctatgaag gactggactg gctgtccaat |
| 601 | cagctccgga accagaagtg aacgcgaccc ccctccctct cactcctctt gccctctgct |
| 661 | ttactctcat gtggcaaacg tgcggctcgt ggtgtgagtg ccagaagctg cctccgtggt |
| 721 | ttggtcaccg tgtgcatcgc accgtgctgt aaatgtggca gacgcagcct gcggccaggc |
| 781 | tttttattta atgtaaatag tttttgtttc caatgaggca gtttctggta ctcctatgca |
| 841 | atattactca gcttttttta ttgtaaaaag aaaaatcaac tcactgttca gtgctgagag |
| 901 | gggatgtagg cccatgggca cctggcctcc aggagtcgct gtgttgggag agccggccac |
| 961 | gcccttggct tagagctgtg ttgaaatcca ttttggtggt tggttttaac ccaaactcag |
| 1021 | tgcatttttt aaaatagtta agaatccaag tcgagaacac ttgaacacac agaagggaga |
| 1081 | ccccgcctag catagatttg cagttacggc ctggatgcca gtcgccagcc cagctgttcc |
| 1141 | cctcgggaac atgaggtggt ggtggcgcag cagactgcga tcaattctgc atggtcacag |
| 1201 | tagagatccc cgcaactcgc ttgtccttgg gtcaccctgc attccatagc catgtgcttg |
| 1261 | tccctgtgct cccacggttc ccaggggcca ggctgggagc ccacagccac cccactatgc |
| 1321 | cgcaggccgc cctacccacc ttcaggcagc ctatgggacg caggccccat ctgtccctcg |
| 1381 | gtccgcgtgt ggccagagtg gtccgtcgtc cccaacactc gtgctcgctc agacactttg |
| 1441 | gcaggatgtc tggggcctca ccagcaggag cgcgtgcaag ccgggcaggc ggtccaccta |
| 1501 | gacccacagc ccctcgggag caccccacct ctgtgtgtga tgtagctttc tctccctcag |
| 1561 | cctgcaaggg tccgatttgc catcgaaaaa gacaacctct acttttttct tttgtatttt |
| 1621 | gataaacact gaagctggag ctgttaaatt tatcttgggg aaacctcaga actggtctat |
| 1681 | ttggtgtcgt aggaacctct tactgctttc aatacacgat tagtaatcaa ctgttttgta |
| 1741 | tacttgtttt cagttttcat ttcgacaaac aagcactgta attatagcta ttagaataaa |
| 1801 | atctcttaac tatt |

SEQ ID NO: 4 human ARF1 polypeptide sequence
MGNIFANLFKGLFGKKEMRILMVGLDAAGKTTILYKLKLGEIVT
TIPTIGFNVETVEYKNISFTVWDVGGQDKIRPLWRHYFQNTQGLIFVVDSNDRERVNE
AREELMRMLAEDELRDAVLLVFANKQDLPNAMNAAEITDKLGLHSLRHRNWYIQATCA
TSGDGLYEGLDWLSNQLRNQK
SEQ ID NO:5 human EPHA2 nucleic acid sequence

| | |
|---|---|
| 1 | cggaagttgc gcgcaggccg gcgggcggga gcggacaccg aggccggcgt gcaggcgtgc |
| 61 | gggtgtgcgg gagccgggct cggggggatc ggaccgagag cgagaagcgc ggcatggagc |
| 121 | tccaggcagc ccgcgcctgc ttcgccctgc tgtggggctg tgcgctggcc gcggccgcgg |
| 181 | cggcgcaggg caaggaagtg gtactgctgg actttgctgc agctggaggg gagctcggct |
| 241 | ggctcacaca cccgtatggc aaagggtggg acctgatgca gaacatcatg aatgacatgc |
| 301 | cgatctacat gtactccgtg tgcaacgtga tgtctggcga ccaggacaac tggctccgca |
| 361 | ccaactgggt gtaccgagga gaggctgagc gtaacaactt tgagctcaac tttactgtac |
| 421 | gtgactgcaa cagcttccct ggtggcgcca gctcctgcaa ggagactttc aacctctact |
| 481 | atgccgagtc ggacctggac tacggcacca acttccagaa gcgcctgttc accaagattg |
| 541 | acaccattgc gcccgatgag atcaccgtca gcagcgactt cgaggcacgc cacgtgaagc |
| 601 | tgaacgtgga ggagcgctcc gtggggccgc tcacccgcaa aggcttctac ctggccttcc |
| 661 | aggatatcgg tgcctgtgtg gcgctgctct ccgtccgtgt ctactacaag aagtgccccg |
| 721 | agctgctgca gggcctggcc cacttccctg agaccatcgc cggctctgat gcaccttccc |
| 781 | tggccactgt ggccggcacc tgtgtggacc atgccgtggt gccaccgggg ggtgaagagc |
| 841 | cccgtatgca ctgtgcagtg gatggcgagt ggctggtgcc cattgggcag tgcctgtgcc |
| 901 | aggcaggcta cgagaaggtg gaggatgcct gccaggcctg ctcgcctgga ttttttaagt |
| 961 | ttgaggcatc tgagagcccc tgcttggagt gccctgagca cacgctgcca tcccctgagg |
| 1021 | gtgccacctc ctgcgagtgt gaggaaggct tcttccgggc acctcaggac ccagcgtcga |
| 1081 | tgccttgcac acgaccccct tccgccccac actacctcac agccgtgggc atgggtgcca |
| 1141 | aggtggagct gcgctggacg ccccctcagg acagcggggg ccgcgaggac attgtctaca |
| 1201 | gcgtcacctg cgaacagtgc tggcccgagt ctggggaatg cgggccgtgt gaggccagtg |
| 1261 | tgcgctactc ggagcctcct cacggactga cccgcaccag tgtgacagtg agcgacctgg |
| 1321 | agccccacat gaactacacc ttcaccgtgg aggcccgcaa tggcgtctca ggcctggtaa |
| 1381 | ccagccgcag cttccgtact gccagtgtca gcatcaacca gacagagccc cccaaggtga |
| 1441 | ggctggaggg ccgcagcacc acctcgctta gcgtctcctg gagcatcccc ccgccgcagc |
| 1501 | agagccgagt gtggaagtac gaggtcactt accgcaagaa gggagactcc aacagctaca |
| 1561 | atgtgcgccg caccgagggt ttctccgtga ccctggacga cctggcccca gacaccacct |
| 1621 | acctggtcca ggtgcaggca ctgacgcagg agggccaggg ggccggcagc aaggtgcacg |
| 1681 | aattccagac gctgtccccg gagggatctg gcaacttggc ggtgattggc ggcgtggctg |
| 1741 | tcggtgtggt cctgcttctg gtgctggcag gagttggctt ctttatccac cgcaggagga |
| 1801 | agaaccagcg tgcccgccag tccccggagg acgtttactt ctccaagtca gaacaactga |
| 1861 | agcccctgaa gacatacgtg gacccccaca catatgagga ccccaaccag gctgtgttga |
| 1921 | agttcactac cgagatccat ccatcctgtg tcactcggca gaaggtgatc ggagcaggag |
| 1981 | agtttgggga ggtgtacaag ggcatgctga agacatcctc ggggaagaag gaggtgccgg |
| 2041 | tggccatcaa gacgctgaaa gccggctaca cagagaagca gcgagtggac ttcctcggcg |
| 2101 | aggccggcat catgggccag ttcagccacc acaacatcat ccgcctagag ggcgtcatct |
| 2161 | ccaaatacaa gcccatgatg atcatcactg agtacatgga gaatggggcc ctggacaagt |
| 2221 | tccttcggga gaaggatggc gagttcagcg tgctgcagct ggtgggcatg ctgcggggca |
| 2281 | tcgcagctgg catgaagtac ctggccaaca tgaactatgt gcaccgtgac ctggctgccc |
| 2341 | gcaacatcct cgtcaacagc aacctggtct gcaaggtgtc tgactttggc ctgtcccgcg |
| 2401 | tgctggagga cgaccccgag gccacctaca ccaccagtgg cggcaagatc cccatccgct |
| 2461 | ggaccgcccc ggaggccatt tcctaccgga agttcacctc tgccagcgac gtgtggagct |
| 2521 | ttggcattgt catgtgggag gtgatgacct atggcgagcg gccctactgg gagttgtcca |
| 2581 | accacgaggt gatgaaagcc atcaatgatg gcttccggct ccccacaccc atggactgcc |
| 2641 | cctccgccat ctaccagctc atgatgcagt gctggcagca ggagcgtgcc cgccgcccca |
| 2701 | agttcgctga catcgtcagc atcctggaca agctcattcg tgcccctgac tccctcaaga |
| 2761 | ccctggctga ctttgacccc cgcgtgtcta tccggctccc cagcacgagc ggctcggagg |
| 2821 | gggtgccctt ccgcacggtg tccgagtggc tggagtccat caagatgcag cagtatacgg |
| 2881 | agcacttcat ggcggccggc tacactgcca tcgagaaggt ggtgcagatg accaacgacg |
| 2941 | acatcaagag gattggggtg cggctgcccg gccaccagaa gcgcatcgcc tacagcctgc |
| 3001 | tgggactcaa ggaccaggtg aacactgtgg ggatccccat ctgagcctcg acagggcctg |
| 3061 | gagccccatc ggccaagaat acttgaagaa acagagtggc ctccctgctg tgccatgctg |
| 3121 | ggccactggg gactttattt atttctagtt ctttcctccc cctgcaactt ccgctgaggg |
| 3181 | gtctcggatg acaccctggc ctgaactgag gagatgacca gggatgctgg gctgggccct |
| 3241 | ctttccctgc gagacgcaca cagctgagca cttagcaggc accgccacgt cccagcatcc |
| 3301 | ctggagcagg agccccgcca cagccttcgg acagacatat aggatattcc caagccgacc |
| 3361 | ttccctccgc cttctcccac atgaggccat ctcaggagat ggagggcttg gcccagcgcc |
| 3421 | aagtaaacag ggtacctcaa gccccatttc ctcacactaa gagggcagac tgtgaacttg |
| 3481 | actgggtgag acccaaagcg gtccctgtcc ctctagtgcc ttctttagac cctcgggccc |
| 3541 | catcctcatc cctgactggc caaacccttg ctttcctggg cctttgcaag atgcttggtt |
| 3601 | gtgttgaggt ttttaaatat atattttgta ctttgtggag agaatgtgtg tgtgtggcag |
| 3661 | ggggccccgc cagggctggg gacagagggt gtcaaacatt cgtgagctgg ggactcaggg |
| 3721 | accggtgctg caggagtgtc ctgcccatgc cccagtcggc cccatctctc atccttttgg |
| 3781 | ataagtttct attctgtcag tgttaaagat tttgttttgt tggacatttt tttcgaatct |
| 3841 | taatttatta ttttttttat atttattgtt agaaaatgac ttatttctgc tctggaataa |
| 3901 | agttgcagat gattcaaacc g |

SEQ ID NO: 6 human EPHA2 polypeptide sequence
MELQAARACFALLWGCALAAAAAAQGKEVVLLDFAAAGGELGWL
THPYGKGWDLMQNIMNDMPIYMYSVCNVMSGDQDNWLRTNWVYRGEAERNNFELNFTV
RDCNSFPGGASSCKETFNLYYAESDLDYGTNFQKRLFTKIDTIAPDEITVSSDFEARH
VKLNVEERSVGPLTRKGFYLAFQDIGACVALLSVRVYYKKCPELLQGLAHFPETIAGS
DAPSLATVAGTCVDHAVVPPGGEEPRMHCAVDGEWLVPIGQCLCQAGYEKVEDACQAC
SPGFFKFEASESPCLECPEHTLPSPEGATSCECEEGFFRAPQDPASMPCTRPPSAPHY
LTAVGMGAKVELRWTPPQDSGGREDIVYSVTCEQCWPESGECGPCEASVRYSEPPHGL
TRTSVTVSDLEPHMNYTFTVEARNGVSGLVTSRSFRTASVSINQTEPPKVRLEGRSTT
SLSVSWSIPPPQQSRVWKYEVTYRKKGDSNSYNVRRTEGFSVTLDDLAPDTTYLVQVQ
ALTQEGQGAGSKVHEFQTLSPEGSGNLAVIGGVAVGVVLLLVLAGVGFFIHRRRKNQR
ARQSPEDVYFSKSEQLKPLKTYVDPHTYEDPNQAVLKFTTEIHPSCVTRQKVIGAGEF
GEVYKGMLKTSSGKKEVPVAIKTLKAGYTEKQRVDFLGEAGIMGQFSHHNIIRLEGVI
SKYKPMMIITEYMENGALDKFLREKDGEFSVLQLVGMLRGIAAGMKYLANMNYVHRDL
AARNILVNSNLVCKVSDFGLSRVLEDDPEATYTTSGGKIPIRWTAPEAISYRKFTSAS
DVWSFGIVMWEVMTYGERPYWELSNHEVMKAINDGFRLPTPMDCPSAIYQLMMQCWQQ
ERARRPKFADIVSILDKLIRAPDSLKTLADFDPRVSIRLPSTSGSEGVPFRTVSEWLE
SIKMQQYTEHFMAAGYTAIEKVVQMTNDDIKRIGVRLPGHQKRIAYSLLGLKDQVNTV
GIPI

1. A method of detecting a breast cancer cell in a biological sample from a patient, the method comprising
contacting the sample with a polynucleotide that selectively hybridizes to a nucleic acid sequence encoding a polypeptide having an amino acid sequence of SEQ ID NO:2, SEQ ID NO:4, or SEQ ID NO:6; and
detecting an increase in the level of the nucleic acid sequence, relative to normal, thereby detecting the presence of a breast cancer in the patient.

2. The method of claim 1, wherein the detecting step comprises detecting 2 an mRNA that encodes the polypeptide.

3. The method of claim 2, wherein the mRNA is detected using an amplification reaction.

4. The method of claim 1, wherein the detecting step comprises detecting an increase in copy number of the nucleic acid that encodes the polypeptide.

5. The method of claim 1, wherein the patient is undergoing a therapeutic regimen to treat breast cancer.

6. The method of claim 1, wherein the patient is suspected of having breast cancer.

7. A method of detecting a breast cancer cell in a biological sample from a patient, the method comprising
detecting an increase in the level of a polypeptide having an amino acid sequence of SEQ ID NO:2, SEQ ID NO:4, or SEQ ID NO:6, relative to normal, thereby detecting the presence of a breast cancer in the patient.

8. The method of claim 7, wherein the step of detecting an increase in the level of the polypeptide comprises performing an immunoassay.

9. A method of monitoring the efficacy of a therapeutic treatment of cancer, the method comprising the steps of:
(i) providing a biological sample from a patient undergoing the therapeutic treatment; and
(ii) detecting the level of: a polypeptide having an amino acid sequence of SEQ ID NO:2, SEQ ID-NO:4, or SEQ ID NO:6, or of a nucleic acid that encodes the polypeptide, in the biological sample compared to a level in a biological sample from the patient prior to, or earlier in, the therapeutic treatment, thereby monitoring the efficacy of the therapy.

10. A method for identifying a compound that modulates a breast cancer-associated polypeptide, the method comprising the steps of:
(i) contacting the compound with a polypeptide of SEQ ID NO:2, SEQ ID NO:4, or SEQ ID NO:6; and
(ii) determining the functional effect of the compound upon the polypeptide.

11. A method of inhibiting proliferation of a breast cancer cell that overexpresses a polypeptide having an amino acid sequence of SEQ ID NO:2, SEQ ID NO:4, or SEQ ID NO:6, the method comprising the step of contacting the cancer cell with a therapeutically effective amount of an inhibitor of the polypeptide.

12. The method of claim 11, wherein the gene that encodes the polypeptide is increased in copy number in the breast cancer cell.

13. The method of claim 11, wherein the inhibitor is an antibody.

14. The method of claim 11, wherein the inhibitor is a small molecule.
